(19) United States
(12) Patent Application Publication
(10) Pub. No.: US 2026/0085295 A1
(43) Pub. Date: Mar. 26, 2026

(54) PARTITIONING ANION EXCHANGE CHROMATOGRAPHY FOR PURIFICATION OF RECOMBINANT ADENO-ASSOCIATED VIRUS (rAAV)

(71) Applicant: Regeneron Pharmaceuticals, Inc., Tarrytown, NY (US)

(72) Inventors: Garima Thakur, White Plains, NY (US); Sheldon Robert Mink, Hawthorne, NY (US); Andrew David Tustian, Millwood, NY (US)

(21) Appl. No.: 19/337,204

(22) Filed: Sep. 23, 2025

Related U.S. Application Data
(60) Provisional application No. 63/697,794, filed on Sep. 23, 2024.

(51) Int. Cl. C12N 7/00; B01D 15/36

(52) U.S. Cl. CPC C12N 7/00; B01D 15/363; C12N 2750/14151

(57) ABSTRACT

The present inventions provide methods for purifying adeno-associated viruses (AAV) that comprise gene of interest (GOI). The present inventions provide partitioning anion-exchange chromatography (AEX), leveraging surface charge differences between empty and full capsids to create an environment of on-column competition for binding sites. This results in empty capsids being removed during the column loading phase, allowing the bulk of full capsids to be eluted in a concentrated, highly enriched pool. The inventions also provide methods for conducting partitioning anion-exchange chromatography in rapid cycling mode for AAV purification process intensification.

This application claims priority to U.S. Ser. No. 63/697,794, filed Sep. 23, 2024, which is hereby incorporated by reference.

### FIELD OF THE INVENTIONS

The present inventions provide systems and methods purifying adeno-associated viruses (AAV) capsids. The inventions provide partitioning anion exchange chromatography (AEX) for AAV manufacturing process intensification. The inventions further provide improved approaches for purification of AAV, such as for the separation of full and empty AAV capsids using partitioning AEX with the option of conducting the operation using rapid cycling.

### BACKGROUND OF THE INVENTIONS

Adeno-associated virus (AAV) is a non-enveloped, single-stranded DNA virus and is used as a gene delivery vector for both research and therapeutics. Weitzman and Linden, Adeno-Associated Virus Biology (chapter 1), Meth. Molec. Biol. 807:1-23 (2011). There are numerous AAV serotypes and variants thereof. AAV serotypes include, for example, AAV1, 2, 3, 4, 5, 6, 7, 8, 9, 10, 11, 12, 13, rh10, rh39, rh43, rh74 as well as variants thereof. AAV serotypes share common properties, structure, and genomic sequence and organization. See, for example, Issa et al, Cells 12:285 (2023); Goedeker et al., Ther. Adv. Neurol. Disord. 16:1-7 (2023).

Gene transfer vectors based on AAV have demonstrated promise for human gene therapy based on their safety profile and potential to achieve long-term efficacy in animal models. Wang et al., Nature, 18:358-78 (2019). A major challenge for advancing AAV-based therapies into clinical development is the difficulty and cost of producing sufficient quantities of AAV through transient methodologies.

Recombinant AAVs (rAAVs) are produced in engineered host cells that contain the requisite genes to allow to produce the recombinant virus. Recombinant AAV has been produced in HEK 293, BHK, human amniotic (for example, epithelial cells such as HAEpiC), CHO, HeLa and Sf9 lines, for example.

The wild type AAV genome includes a capsid gene referred to as “Cap” or “cap.” Cap in nature is translated to produce, via alternative start codons and transcript splicing, three size-variant structural proteins referred to as VP1 (about 90 kDa), VP2 (about 72 kDa) and VP3 (about 60 kDa). An AAV capsid contains 60 subunits total of the VP proteins. A ratio of 1:1:10 is considered the most typical ratio for VP1: VP2: VP3, with a stoichiometry of 5 VP1 subunits: 5 VP2 subunits: 50 VP3 subunits. However, there can be variation. Wörner et al., Nature Communications 12:1642 (2021). Additionally, production of recombinant AAV results in capsids that are empty, nearly empty, or only partially filled in terms of a complete gene of interest and flanking ITRs, which are considered impurities and often are collectively referred to in the field as empty capsids.

Additionally, separation of full capsids from empty capsids, nearly empty capsids, and partially-filled capsids to lessen the presence of these unfilled capsids is an important step in manufacturing of recombinant AAV. Current cell culture technology does not produce only full capsids, which are capsids that contain the entire gene of interest (GOI) and functional flanking AAV inverted terminal repeats (ITRs) required to produce the desired therapeutic effect in vivo. Only a percentage of the rAAV capsids produced in cell culture are full, while the others are partially-filled, nearly empty or completely empty. It often can be difficult to distinguish between full and partially-filled capsids if the partially-filled capsids are close to full. Likewise, it can be difficult to distinguish between nearly empty and empty capsids if the nearly empty capsids are close to empty. Thus, there are degrees of capsid contents that are less than a complete GOI and flanking ITRs, but nevertheless still are not full. Capsids that are not full (in other words, less than full) include partially filled capsids, nearly empty capsids, and/or empty capsids. The goal is to enrich for full capsids, and thereby functional capsids, and minimize capsids that are less than full and therefore will not be functional, which is achieved by the present inventions.

Having a high percentage of empty capsids, nearly empty capsids and partially filled capsids in the drug product is undesirable as they contribute to the immune response produced upon drug administration without adding any therapeutic benefit because such capsids do not contain a functional gene of interest and/or functional flanking ITRs. Thus, it is critical to enrich for full capsids in the downstream purification process to deliver safer and more efficacious drug products to patients.

### SUMMARY OF THE INVENTIONS

The inventions advantageously employ partitioning chromatography methods that involve loading of a chromatographic unit such that all accessible binding sites are taken up by species in the load material, after which tighter-binding species displace weaker-binding species due to competition for the binding sites, removing weaker-binding species in the flowthrough. For most AAV serotypes, full capsids bind tighter than capsids that are not full (for example, empty capsids). However, with certain AAVs, such as AAV1 and AAV9, the opposite can occur; some capsids that are not full (for example, empty capsids) can bind tighter than full capsids.

The inventions provide improved methods of partitioning Anion Exchange Chromatography (AEX) for separation of full capsids from not full AAV capsids in order to provide efficacious recombinant AAV preparations. Distinction between full capsids from not full capsids can be determined using, for example, mass photometry, analytical centrifugation (AUC) and techniques to measure the genomic titer relative to the capsid titer, such as polymerase chain reaction (PCR) and capsid enzyme-linked immunosorbent assays (ELISA).

The inventions advantageously provide improved methods of partitioning Anion Exchange Chromatography (AEX) for separation of full and not full capsids, such as partially filled, nearly empty and/or empty capsids of AAV. The inventions provide methods of purifying preparations of recombinant adeno-associated virus (rAAV) particles that comprise a gene of interest (GOI), wherein the method comprises the steps of: (a) loading the preparation comprising rAAV viral particles on anion exchange chromatography column (AEX), wherein prior to loading the preparation is mixed with a load buffer at pH of about 6.0 to 11.0, and continue the loading beyond the AEX column binding capacity, wherein the load amount is between about 1×10¹⁵ to 4×10¹⁵ AAV capsids/milliliter (cp/ml) (alternative exponent nomenclature would be 1e15 to 4e15) of AEX column volume; (b) changing the conductivity in the column to about 2 to 12 millisiemens/centimeter (mS/cm); (c) eluting the column with an elution buffer, wherein the elution buffer comprises the load buffer and at least one salt to increase the conductivity of the elution buffer relative to the load buffer; and (d) collecting separated fractions of full and not full rAAV capsids. According to the inventions, the conductivity and pH of the preparation to be loaded can be adjusted prior to loading on the column. The AEX can be conducted in multiple cycles with rapid flow rates of about 1 to 10 column volume/minute (CV/min), about 1 to 9 CV/min, about 1 to 8 CV/min, about 1 to 7 CV/min, about 1 to 6 CV/min, about 1 to 5 CV/min, about 1 to 4 CV/min, about 1 to 3 CV/min, or about 1 to 2 CV/min. The AEX can be conducted in multiple cycles with rapid flow rates of about 1 CV/min, 2 CV/min, 3 CV/min, 4 CV/min, 5 CV/min, 6 CV/min, 7 CV/min, 8 CV/min, 9 CV/min, or 10 CV/min. The pH of the load buffer can be about 6.0, 6.5, 7.0, 7.5, 8.0, 8.5, 9.0, 9.5, 1.0, 10.5, or 11.0. The pH of the load buffer can be about 6.0 to 10, about 6.0 to 9.5, about 6.0 to 9, about 6.0 to 8.5, about 6.0 to 8, about 6.0 to 7.5, about 6.0 to 7, or about 6.0 to 6.5. The final load conductivity can be about 2 mS/cm, about 3 mS/cm, about 4 mS/cm, about 5 mS/cm, about 6 mS/cm, about 7 mS/cm, about 8 mS/cm, about 9 mS/cm, about 10 mS/cm, or about 11 mS/cm. The final load conductivity can be about 2 mS/cm to about 11 mS/cm, about 2 mS/cm to about 10 mS/cm, about 2 mS/cm to about 9 mS/cm, about 2 mS/cm to about 8 mS/cm, about 2 mS/cm to about 7 mS/cm, about 2 mS/cm to about 6 mS/cm, about 2 mS/cm to about 5 mS/cm, about 2 mS/cm to about 4 mS/cm, or 2 mS/cm to about 3 mS/cm. The genomic yield can be about 20%, 25%, 30%, 35%, 40%, 45%, 50%, 55%, 60%, 65%, 70%, 75%, 80%, 85%, 90% or more with VC of 65% VC, 70% VC, 75% VC, 80% VC, 85% VC, 90% VC or more in the separated fractions. The genomic yield can be about 20% to 90%, 20% to 85%, 20% to 80%, 20% to 75%, 20% to 70%, 20% to 65%, 20% to 60%, 20% to 55%, 20% to 50%, 20% to 45%, 20% to 40%, 20% to 35%, 20% to 30%, 20% to 25%, with VC of 65% to 90% VC, 65% to 85% VC, 65% to 80% VC, 65% to 75% VC, 65% to 70% VC in the separated fractions. The rAAV viral particles are loaded on at least one AEX column. The AEX column unit can be selected from the group consisting of monoliths, resins, gels, and membranes. The salt in the elution buffer can be NaCl, KCl, MgCl₂, CaCl₂, NH₄Cl, Na₂SO₄, CaSO₄, K₂SO₄, MgSO₄, (NH₄)₂SO₄, sodium citrate, tetramethylammonium chloride (TMAC) or a mixture thereof.

The inventions also provide methods of separating full capsids and empty capsids in preparations of recombinant adeno-associated virus (rAAV) particles that comprise a gene of interest (GOI), wherein the method comprises the steps of: (a) loading the preparation comprising rAAV viral particles on anion exchange chromatography column (AEX), wherein prior to loading the preparation is mixed with a load buffer at pH of about 6.0 to 11, and continue the loading beyond the AEX column binding capacity until some capsids flow out of the column during continued inflow of load material, wherein the load amount is between about 1×10¹⁵ to 6×10¹⁵ cp/ml; (c) changing the conductivity in the column to about 2 to 12 mS/cm; (d) eluting the column with an elution buffer, wherein the elution buffer comprises the load buffer and at least one salt to increase the conductivity of the elution buffer relative to the load buffer; and (e) collecting separated fractions of full and not full rAAV capsids eluted using either linear gradient or isocratic elutions. According to the inventions, the load buffer conductivity and pH can be adjusted prior to loading the preparation. The AEX can be conducted in multiple cycles with rapid flow rates of about 1 to 10 CV/min, about 1 to 9 CV/min, about 1 to 8 CV/min, about 1 to 7 CV/min, about 1 to 6 CV/min, about 1 to 5 CV/min, about 1 to 4 CV/min, about 1 to 3 CV/min, or about 1 to 2 CV/min. The AEX can be conducted in multiple cycles with rapid flow rates of about 1 CV/min, 2 CV/min, 3 CV/min, 4 CV/min, 5 CV/min, 6 CV/min, 7 CV/min, 8 CV/min, 9 CV/min, or 10 CV/min. The pH of the load buffer can be about 6.0, 6.5, 7.0, 7.5, 8.0, 8.5, 9.0, 9.5, 1.0, 10.5, or 11.0. The pH of the load buffer can be about 6.0 to 10, about 6.0 to 9.5, about 6.0 to 9, about 6.0 to 8.5, about 6.0 to 8, about 6.0 to 7.5, about 6.0 to 7, or about 6.0 to 6.5. The final load conductivity can be about 2 mS/cm, about 3 mS/cm, about 4 mS/cm, about 5 mS/cm, about 5 mS/cm, about 7 mS/cm, about 8 mS/cm, about 9 mS/cm, about 10 mS/cm, or about 11 mS/cm. The final load conductivity can be about 2 mS/cm to about 11 mS/cm, about 2 mS/cm to about 10 mS/cm, about 2 mS/cm to about 9 mS/cm, about 2 mS/cm to about 8 mS/cm, about 2 mS/cm to about 7 mS/cm, about 2 mS/cm to about 6 mS/cm, about 2 mS/cm to about 5 mS/cm, about 2 mS/cm to about 4 mS/cm, or 2 mS/cm to about 3 mS/cm. The genomic yield can be about 20%, 25%, 30%, 35%, 40%, 45%, 50%, 55%, 60%, 65%, 70%, 75%, 80%, 85%, 90% or more with VC of 65% VC, 70% VC, 75% VC, 80% VC, 85% VC, 90% VC, or more in the separated fractions. The genomic yield can be about 20% to 90%, 20% to 85%, 20% to 80%, 20% to 75%, 20% to 70%, 20% to 65%, 20% to 60%, 20% to 55%, 20% to 50%, 20% to 45%, 20% to 40%, 20% to 35%, 20% to 30%, 20% to 25%, with VC of 65% to 90% VC, 65% to 85% VC, 65% to 80% VC, 65% to 75% VC, 65% to 70% VC in the separated fractions. The rAAV viral particles can be loaded on at least one AEX column. The AEX column unit can be selected from the group consisting of monoliths, resins, gels, and membranes. The salt in the elution buffer can be NaCl, KCl, MgCl₂, CaCl₂, NH₄Cl, Na₂SO₄, CaSO₄, K₂SO₄, MgSO₄, (NH₄)₂SO₄, sodium citrate, tetramethylammonium chloride (TMAC) or a mixture thereof.

The inventions also provide rAAV, rAAV preparations produced by any of the above the methods, and drug products made from the rAAV preparations.

The inventions are amendable to use with all AAV serotypes and variants, including but not limited to AAV1, AAV2, AAV2.7m8, AAV2quad(Y-F), AAV3, AAV4, AAV5, AAV6, AAV7, AAV8, AAV9, AAV10, AAV11, AAV12, AAV13, AAV rh10, AAV rh39, AAV rh43, AAV rh74 and any variants thereof. The inventions are also amenable to use with surface-modified AAV capsids (for example, AAV capsids comprising SpyTag amino acid sequences).

### BRIEF DESCRIPTION OF THE FIGURES

FIG. 1 is a schematic depiction showing the effect of loading on breakthrough of empty and full capsids and full/empty capsid ratio in the flowthrough.

FIG. 2A schematically depicts standard bind-and-elute AEX that is traditionally conducted in a linear gradient mode. FIG. 2B schematically depicts partitioning AEX used according to the inventions.

FIGS. 3A and 3B illustrate the impacts of loading amount and conductivity on capsid displacement effects.

FIG. 4 shows capsid titer and mass photometry analysis of breakthrough fractions, illustrating that empty capsids are displaced by full capsids during continued application of load material beyond column binding capacity when the pH and conductivity of the load material is within a range suitable for displacement effects.

FIG. 5 is a schematic depiction showing linear gradient elution graphs and the effect of loading in achieving an appropriate amount of displacement for the separation of empty and full capsids.

FIG. 6 shows percent (%) full capsids compared to bind-and-elute process for loadings inside the displacement zone.

FIGS. 7A and 7B showing UV260/280 ratio of capsid breakthrough profile (FIG. 7A) and Mass photometry histogram of elution pool (FIG. 7B). Mass photometry can detect weight differences of capsids.

FIGS. 8A to 8C show the effects of rapid cycling displacement on monolith volume, associated cost, and buffer volume, wherein a small column volume is used such that the load material can be processed in about 10 cycles instead of 1 cycle. Rapid cycling displacement chromatography: (i) can lower monolith volume by about 100-fold (FIG. 8A); (ii) can lower estimated associated cost by about 40-fold for 500L bioreactor and about 60-fold for a 50L bioreactor FIG. 8B); and (iii) can lower buffer volume by about 10-fold (FIG. 8C).

FIG. 9 illustrates that continuous processing approaches can be used to intensify AAV purification processes.

FIG. 10 depicts production purification trains. The top train uses a batch tangential flow filtration unit that takes four days. The bottom train replaces the batch tangential flow filtration (TFF) unit with a single-pass tangential flow filtration (SPTFF) unit and takes two days. Ionic exchange chromatography of different modalities can be used following TFF, and anion exchange is depicted as an exemplar. Continuous processing techniques including SPTFF and multi-column or rapid-cycling chromatography also can be applied.

FIGS. 11A to 11F are schematic depictions from monitoring AEX flowthrough using UV260 and UV280 signals during overloading of AAV8 capsids on CIM QA monolith at load conductivities of 2 (FIG. 11A), 4 (FIG. 11B), 6 (FIG. 11C), 8 (FIG. 11D), and 10 (FIG. 11E) mS/cm, showing weak partitioning effects for FIG. 11C and FIG. 11D. FIG. 11F is an offline mass photometry analysis of flowthrough fractions from FIG. 11D.

FIG. 12 illustrates a weak partitioning AEX chromatogram showing calculations of L_{E} (capsid loading required to begin breakthrough of Empty Capsids (EC) species), L_{F} (capsid loading required to begin breakthrough of Viral Capsids (VC) species (capsids containing genetic material, mostly full capsids)) and k_{WP} (weak partitioning coefficient) and elution using conductivity increments of 1 mS/cm.

FIGS. 13A to 13D show AEX elution pool percentage (%) VC (FIG. 13A) and genomic yield (FIG. 13B), along with weak partitioning AEX parameters partitioning coefficient k_{WP} (FIG. 13C) monolith loading until VC breakthrough (FIG. 13D), for AEX runs conducted with different load pH and [Mg²⁺].

FIGS. 14A to 14E show AEX elution pool % VC (FIG. 14A), partitioning coefficient k_{WP} (FIG. 14B), and genomic yield (FIG. 14C) for AEX runs conducted with different starting % VC in the load material at constant pH of 8.5. The impact of starting % VC on k_{WP} is illustrated further through (FIG. 14D) and (FIG. 14E) comparing loads with 8% and 65% VC, respectively.

FIGS. 15A to 15C are graphs showing that loading conductivity correlates with elution conductivity for weak partitioning AEX (FIGS. 15B and 15C), unlike in the case of standard AEX without weak partitioning (FIG. 15A).

FIG. 16 is a schematic depiction of strategies for weak partitioning AEX with isocratic elution, using a single high-salt elution buffer with gradient pump to control both load dilution as well as elution, resulting in a process suitable for large-scale operations that is robust within conductivity variations of ranging from about 9-11 mS/cm for the buffer.

FIGS. 17A to 17E illustrate chromatograms of weak partitioning AEX with isocratic elution using UV260/280 ratio triggers of (FIG. 17A) 1.0, (FIG. 17B) 1.1 and (FIG. 17C) 1.3 to end the wash phase; (FIG. 17D) Impact on % VC and % genomic yield in the isocratic elution pool; (FIG. 17E) Comparable UV260/280 ratio profiles during weak partitioning load for four different CIM QA SpeCIMen units sourced from parent monoliths.

FIGS. 18A to 18F show results from rapid-cycling weak partitioning AEX across 10 cycles of operation (FIG. 18A) with process variations in elution buffer conductivity (FIG. 18B), load % VC (FIG. 18C) and load capsid titer (FIG. 18D), resulting in consistently >75% genomic yield (FIG. 18E) and >80% VC (FIG. 18F) in the elution pools for the 3-hour rapid cycling process.

FIGS. 19A to 19J show comparison of operating parameters, including monolith volume needed (FIGS. 19A-19B), monolith loading per cycle (FIGS. 19C-19D), number of cycles (FIGS. 19E-19F), buffer volume needed (FIGS. 19G-19H), and total operating time (FIGS. 19I-19J) to process 500 L scale bioreactors with titers in the range of 1e12 to 1e14 cp/mL using linear gradient operation (FIGS. 19A, 19C, 19E, 19G and 19I) compared to rapid cycling weak partitioning AEX (FIGS. 19B, 19D, 19F, 19H and 19J).

FIGS. 20A to 20B depict the results of rapid-cycling chromatography process. Overlay of loading, wash, elution, and strip phases of 20 cycles of partitioning AEX on 1 mL CIM QA HR monolith are shown in FIG. 20A. FIG. 20B shows % Full and % Partial capsids in partitioning AEX pool tracked over 42 hours of rapid cycling operation.

### DETAILED DESCRIPTION OF THE INVENTIONS

### Definitions

Unless defined otherwise, all technical and scientific terms used herein have the same meaning as commonly understood by one of ordinary skill in the art to which this invention belongs.

The term “about” in the context of numerical values and ranges refers to values or ranges that approximate or are close to the recited values or ranges such that the invention can perform as intended, such as having a desired number, value, rate, amount, density, degree, increase, decrease, percentage, ratio, value, purity, pH, concentration, presence of a form or variant, temperature or amount of time, as is apparent from the teachings contained herein. For example, “about” can signify values either above or below the stated value in a range of approximately +/−10% or more or less depending on the ability to perform. Thus, this term encompasses values beyond those simply resulting from systematic error.

“Polynucleotide” includes a sequence of nucleotides covalently joined and includes RNA and DNA. Oligonucleotides are considered shorter polynucleotides. Genes are DNA polynucleotides (polydeoxyribonucleic acid) that ultimately encode polypeptides, which are translated from RNA (polyribonucleic acid) that was typically transcribed from DNA. DNA polynucleotides also can encode RNA polynucleotides that is not translated, but rather function as RNA “products.” The type of polynucleotide (that is, DNA or RNA) is apparent from the context of the usage of the term. A polynucleotide referred to or identified by the polypeptide it encodes sets forth and covers all suitable sequences in accordance with codon degeneracy. Polynucleotides, including those disclosed herein, include percent identity sequences and homologous sequences when indicated.

“Polypeptide” or “peptide” refers to sequence(s) of amino acids covalently joined. Polypeptides include natural, semi-synthetic and synthetic proteins and protein fragments. “Polypeptide” and “protein” can be used interchangeably. Oligopeptides are considered shorter polypeptides.

A “gene of interest” (GOI) encodes a “protein of interest.” “Protein of interest” or “polypeptide of interest” (POI) can have any amino acid sequence, and includes any protein, polypeptide, or peptide that is desired to be expressed. Included are, but not limited to, viral proteins, bacterial proteins, fungal proteins, plant proteins and animal (including human) proteins. Protein types can include, but are not limited to, antibodies (including derivatives and fragment, receptors, Fc-containing proteins, trap proteins (including mini-trap proteins), fusion proteins, antagonists, inhibitors, enzymes (such as those used in enzyme replacement therapy), factors, repressors, activators, ligands, reporter proteins, selection proteins, protein hormones, protein toxins, structural proteins, storage proteins, transport proteins, neurotransmitters and contractile proteins. Derivatives, components, domains, chains, and fragments of the above also are included. The sequences can be natural, semi-synthetic or synthetic. Native sequences, mutant sequences and degenerate sequences can be GOls. A gene of interest also can be referred to as a “transgene.”

A “nucleotide of interest” includes GOls and sequences encoding non-translated RNAs/non-coding RNAs (such as, but not limited to, antisense RNA, small interfering RNA, micro-RNA, catalytic RNA, and ribozymes). NOls and GOIs also can be referred to as “payloads.”

“Protein of interest” or “polypeptide of interest” (POI) can have any amino acid sequence, and includes any protein, polypeptide, or peptide that is desired to be expressed, typically for gene therapy purposes. Protein types can include, but are not limited to, receptors, fusion proteins, agonists, antagonists, activators, inhibitors, enzymes (such as those used in enzyme replacement therapy), factors and co-factors, repressors, activators, ligands, protein hormones, therapeutic proteins, suicide proteins, structural proteins, storage proteins, transport proteins, signal proteins, neurotransmitters and contractile proteins. Derivatives, components, domains, chains, and fragments of the above also are included. The sequences can be natural, semi-synthetic or synthetic.

“Purification” in its various grammatical forms includes, but is not limited to, the use of one or more procedures such as depth filtration, tangential flow filtration, affinity capture, ionic exchange, and the like.

“Rapid cycling” refers to the cyclical loading a chromatography column with multiple column volumes of an AAV preparation per minute. For example, the loaded volumes can range from about 2 to 20 times (for example, about 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, 20) the volume of the column per minute, more preferably range from about 3 to 10 times (for example, about 3, 3.5, 4, 4.5, 5, 5.5, 6, 6.5, 7, 7.5, 8, 8.5, 9, 9.5, 10) the volume of the column per minute. For example, with rapid cycling a 500 mL AEX column can be preferably cycled with a range of about 1.5L to 5L of an AAV preparation per minute.

All numerical limits and ranges set forth herein include all numbers or values thereabout or there between of the numbers of the range or limit. The ranges and limits described herein expressly denominate and set forth all integers, decimals and fractional values defined and encompassed by the range or limit. Thus, a recitation of ranges of values herein are intended to serve as a way of referring individually to each separate value falling within the range, unless otherwise indicated herein, and each separate value is incorporated into the specification as if it were individually recited herein.

### DESCRIPTION

### Rapid Cycling Weak Partitioning Chromatography

For rapid cycling weak partitioning chromatography (also referred to as rapid cycling displacement chromatography), a column is selected of 10- to 100-fold smaller size than is typically be selected to process the total amount of desired material. For example, a 500 L purification of AAV typically use a 4000 mL AEX column in standard bind-and-elute mode without rapid cycling. In rapid cycling weak partitioning chromatography, the column selected can be 40 or 400 mL.

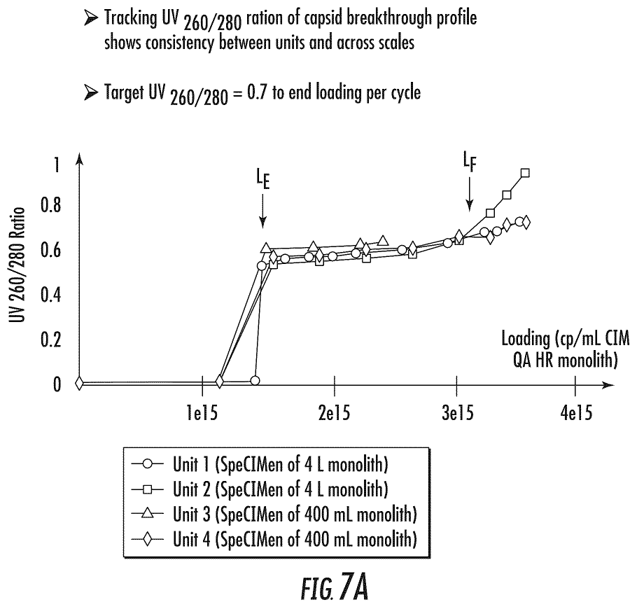

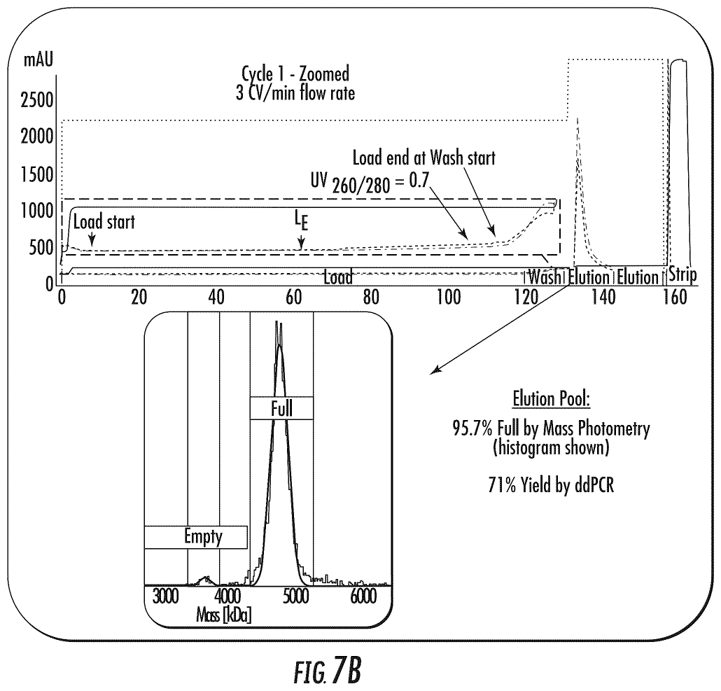

The preparation material is loaded onto the column, and the UV260 and UV280 profile at the outlet of the column is monitored during the load. When a certain UV260/280 threshold is reached (for example, 0.7 as shown in FIG. 7A), sufficient partitioning is completed between the empty and full capsids, and the loading would be stopped. After this, elution is conducted, followed by loading again. These cycles are repeated until the total amount of material is processed, or the lifetime of the column (e.g., >30 cycles) is completed in the case of a continuous process, after which the column is switched to a new column.

The flow rate of the rapid cycling operation can be about 2 to 10 times faster than the flow rate during standard operation (with only one cycle on a larger column). This flow rate is not possible to achieve if the larger column is used (for example, 4000 mL) due to pressure limitations, but can be achieved on the smaller columns in rapid cycling mode.

The inventions provide methods of purifying rAAV capsids using AEX column units. According to the inventions, the AEX is subject to a rapid cycling weak partitioning chromatography. The load buffer can comprise at least one buffering agent, for example Bis-Tris-Propane (BTP), Bis-Tris, Tris, Glycine, Bicine, Tricine, Acetate, Borate, Citrate, Carbonate, Phosphate, Formate, Sulfate, Succinic acid, Sulfonic acid and variants thereof (for example MES, PIPES, HEPES, CHES, CAPS, MMS, PBMS), Diethanolamine, or Imidazole. The buffer can also comprise one or more organic or inorganic salts, such as NaCl, KCl, MgCl₂, CaCl₂, NH₄Cl, Na₂SO₄, CaSO₄, K₂SO₄, MgSO₄, (NH₄)₂SO₄, sodium citrate, and tetramethylammonium chloride (TMAC), or a mixture thereof. The buffer can also comprise additives such as antibiotics and bacteriostatics (for example sodium azide or AEBSF), protease inhibitors (for example E64), detergents, surfactants, and chaotropes (for example poloxamer, CHAPS, SDS, Triton, Tween, Urea), saturating agents (for example bovine serum albumin), organic solvents (for example ethanol, isopropanol, acetonitrile), and other additives or excipients including chelatants, stabilizers, reducers (for example DTT, TCEP, EDTA, Glycerol, Sucrose), and amino acids (for example Histidine) The inventions are amenable to the use of anion exchange (AEX) monoliths (for example CIM QA, CIM DEAE, and PRIMA T), AEX resins (for example Capto Q, CAPTO DEAE, POROS HQ, POROS XQ, POROS PI, Fractogel EMD TMAE, Fractogel EMD DEAE, Nuvia Q), and AEX membranes (for example Sartobind STIC PA, Sartobind Q). The elution buffer can comprise the load buffer and additional NaCl or other salts. The rAAV viral particles are loaded on at least one AEX column.

### Buffers

The load and the elution buffer can comprise at least one buffering agent, including but not limited to Bis-Tris-Propane (BTP), Bis-Tris, Tris, Glycine, Bicine, Tricine, Acetate, Borate, Citrate, Carbonate, Phosphate, Formate, Sulfate, Succinic acid, Sulfonic acid and variants thereof (for example MES, PIPES, HEPES, CHES, CAPS, MMS, PBMS), Diethanolamine, and Imidazole.

The preparation of the rAAV viral particles can comprise about 1 mM to about 50 mM, about 5 mM to about 15 mM, about 10 mM to about 15 mM, about 15 mM to about 20 mM, about 20 mM to about 25 mM, about 25 mM to about 30 mM, about 30 mM to about 35 mM, about 35 mM to about 40 mM, about 40 mM to about 45 mM, about 45 mM to about 50 mM, or about 20 mM+1 mM buffering agent, for example BTP. The preparation can comprise about 5 mM, about 10 mM, about 15 mM, about 20 mM, about 25 mM, about 30 mM, about 35 mM, about 40 mM, about 45 mM, or about 50 mM buffering agent, for example BTP. The preparation can comprise about 5 mM, about 10 mM, about 15 mM, about 20 mM, about 25 mM, about 30 mM, about 35 mM, about 40 mM, about 45 mM, or about 50 mM BTP buffering agent. The preparation also can comprise about 20 mM±2 mM BTP buffer.

### Load Buffer Conductivity

The load buffer conductivity can be adjusted to about 2 to about 12 mS/cm, about 2 to about 11 mS/cm, about 2 to about 10 mS/cm, about 2 to about 9 mS/cm, about 2 to about 8 mS/cm, about 2 to about 7 mS/cm, about 2 to about 6 mS/cm, about 2 to about 5 mS/cm, about 2 to about 4 mS/cm, about 2 to about 3 mS/cm, about 3 to about 11 mS/cm, about 3 to about 10 mS/cm, about 3 to about 9 mS/cm, about 3 to about 8 mS/cm, about 3 to about 7 mS/cm, about 3 to about 6 mS/cm, about 3 to about 5 mS/cm, about 3 to about 4 mS/cm, about 4 to about 11 mS/cm, about 4 to about 10 mS/cm, about 4 to about 9 mS/cm, about 4 to about 8 mS/cm, about 4 to about 7 mS/cm, about 4 to about 6 mS/cm, about 4 to about 5 mS/cm, about 5 to about 11 mS/cm, about 5 to about 10 mS/cm, about 5 to about 9 mS/cm, about 5 to about 8 mS/cm, about 5 to about 7 mS/cm, about 5 to about 6 mS/cm, about 6 to about 11 mS/cm, about 6 to about 10 mS/cm, about 6 to about 9 mS/cm, about 6 to about 8 mS/cm, about 6 to about 7 mS/cm, about 7 to about 11 mS/cm, about 7 to about 10 mS/cm, about 7 to about 9 mS/cm, about 7 to about 8 mS/cm, about 8 to about 11 mS/cm, about 8 to about 10 mS/cm, about 8 to about 9 mS/cm, about 9 to about 11 mS/cm, about 9 to about 10 mS/cm, about 2 mS/cm, about 2.5 mS/cm, about 3 mS/cm, about 3.5 mS/cm, about 4 mS/cm, about 4.5 mS/cm, about 5 mS/cm, about 5.5 mS/cm, about 6 mS/cm, about 6.5 mS/cm, about 7 mS/cm, about 7.5 mS/cm, about 8 mS/cm, about 8.5, about 9 mS/cm, about 9.5 mS/cm, about 10 mS/cm, about 10.5 mS/cm, about 11 mS/cm, about 11.5 mS/cm, or about 12 mS/cm.

### Salts

The salt in the elution buffer can be any organic or inorganic salt, include, but are not limited to, metal salts such as sodium, potassium and cesium salts; alkaline earth metal salts such as calcium and magnesium salts; organic amine salts such as triethylamine, guanidine and N-substituted guanidine salts, acetamidine and N-substituted acetamidine, pyridine, picoline, ethanolamine, triethanolamine, dicyclohexylamine, and N,N′-dibenzylethylenediamine salts. Suitable salts (of basic nitrogen centers) include, but are not limited to inorganic acid salts such as the hydrochloride, hydrobromide, sulfate, phosphate; organic acid salts such as trifluoroacetate and maleate salts; sulfonates such as methanesulfonate, ethanesulfonate, benzenesulfonate, p-toluenesulfonate, camphor sulfonate and naphthalenesulfonate; amino acid salts such as arginate, alaninate, asparginate and glutamate; and carbohydrate salts such as gluconate and galacturonate. Non-limiting examples of acceptable salts include, without limitation, sodium salts, ammonium salts, potassium salts, calcium salts, and magnesium salts (e.g., sodium, ammonium, potassium, calcium, and magnesium chloride; sodium, ammonium, potassium, calcium and magnesium acetate; sodium, ammonium, potassium, calcium and magnesium citrate; sodium, ammonium, potassium, calcium and magnesium phosphate; sodium, ammonium, potassium, calcium and magnesium fluoride; sodium, ammonium, potassium, calcium and magnesium bromide; and sodium, ammonium, potassium, calcium and magnesium iodide). The acceptable salt can be sodium chloride or arginine hydrochloride (L-arginine hydrochloride).

The concentration of the salt in the elution buffer can be about 50 mM to about 500 mM, about 50 mM to about 400 mM, about 50 mM to about 350 mM, about 50 mM to about 300 mM, about 50 mM to about 250 mM, about 50 mM to about 200 mM, about 50 mM to about 150 mM, about 50 mM to about 100 mM, about 50 mM, about 100 mM, about 150 mM, about 200 mM, about 250 mM, about 300 mM, about 350 mM, about 400 mM, about 450 mM, or about 500 mM.

The concentration of the salt in the elution buffer can be about 100±10 mM, about 150±10 mM, about 200±10 mM, about 250±10 mM, about 300±10 mM, about 350±10 mM, about 400±10 mM, about 450±10 mM, about 100±20 mM, about 150±20 mM, about 200±20 mM, about 250±20 mM, about 300±20 mM, about 350±20 mM, about 400±20 mM, or about 450±20 mM.

pH

The pH of the load buffer can be about 6.0 to about 11.0, about 6.0 to about 10.5, about 6.0 to about 10.0, about 6.0 to about 9.5, about 6.0 to about 9.0, about 6.0 to about 8.5, about 6.0 to about 8.0, about 6.0 to about 7.5, about 6.0 to about 7.0, about 6.0 to about 6.5, about 6.5 to about 10.5, about 6.5 to about 10.0, about 6.5 to about 9.5, about 6.5 to about 9.0, about 6.5 to about 8.5, about 6.5 to about 8.0, about 6.5 to about 7.5, about 6.5 to about 7.0, about 7.0 to about 10.5, about 7.0 to about 10.0, about 7.0 to about 9.5, about 7.0 to about 9.0, about 7.0 to about 8.5, about 7.0 to about 8.0, about 7.0 to about 7.5, about 7.5 to about 10.5, about 7.5 to about 10.0, about 7.5 to about 9.5, about 7.5 to about 9.0, about 7.5 to about 8.5, about 7.5 to about 8.0, about 8.5 to about 10.5, about 8.5 to about 10.0, about 8.5 to about 9.5, about 8.5 to about 9.0, about 9.0 to about 10.5, about 9.0 to about 10.0, about 9.0 to about 9.5, about 9.5 to about 10.5, about 9.5 to about 10.0, about 10.0 to about 11.0, about 10.0 to about 10.5, or about 10.5 to about 11.0. The pH of the load buffer can be about 6.0, about 6.5, about 7.0, about 7.5, about 8.0, about 8.5, about 9.0, about 9.5, about 10.0, about 10.5, or about 11.0. The pH of the load buffer can be about 6.0±0.1, about 6.0±0.05, about 7.0±0.1, about 7.0±0.05, about 7.5±0.1, about 7.5±0.05, about 8.0±0.1, about 8.0±0.05, about 8.5±0.1, about 8.5±0.05, about 9.0±0.1, about 9.0±0.05, about 9.5±0.1, about 9.5±0.05, about 10.0±0.1, about 10.0±0.05, about 10.5±0.1, about 10.5±0.05 about 11.0±0.1, or about 11.0±0.05.

### Surfactants

The load buffer of the present disclosure can comprise from about 0.001% (w/v) to about 0.3% (w/v) non-ionic surfactant, such as poloxamers (e.g., poloxamer 188 (P188)).

The load buffer of the present disclosure can comprises non-ionic surfactant, such as poloxamers (e.g., poloxamer 188 (P188)), from about 0.001% (w/v) to about 0.0015% (w/v), about 0.002% (w/v) to about 0.0025% (w/v), about 0.003% (w/v) to about 0.0035% (w/v), about 0.004% (w/v) to about 0.0045% (w/v), about 0.005% (w/v) to about 0.0055% (w/v), about 0.006% (w/v) to about 0.0065% (w/v), about 0.007% (w/v) to about 0.0075% (w/v), about 0.008% (w/v) to about 0.0085% (w/v), about 0.009% (w/v) to about 0.0095% (w/v), about 0.01% (w/v) to about 0.015% (w/v), about 0.02% (w/v) to about 0.025% (w/v), about 0.03% (w/v) to about 0.035% (w/v), about 0.04% (w/v) to about 0.045% (w/v), about 0.05% (w/v) to about 0.055% (w/v), about 0.06% (w/v) to about 0.065% (w/v), about 0.07% (w/v) to about 0.075% (w/v), about 0.08% (w/v) to about 0.085% (w/v), about 0.09% (w/v) to about 0.095% (w/v), about 0.095% (w/v) to about 0.10% (w/v), or any amount therebetween.

The load buffer of the present disclosure can comprises non-ionic surfactant, such as poloxamers (e.g., poloxamer 188 (P188)), from about 0.001% (w/v) to about 0.002% (w/v), about 0.002% (w/v) to about 0.003% (w/v), about 0.003% (w/v) to about 0.004% (w/v), about 0.004% (w/v) to about 0.005% (w/v), about 0.005% (w/v) to about 0.006% (w/v), about 0.006% (w/v) to about 0.007% (w/v), about 0.007% (w/v) to about 0.008% (w/v), about 0.008% (w/v) to about 0.009% (w/v), about 0.009% (w/v) to about 0.01% (w/v), 0.01% (w/v) to about 0.02% (w/v), 0.02% (w/v) to about 0.03% (w/v), 0.03% (w/v) to about 0.04% (w/v), 0.04% (w/v) to about 0.05% (w/v), 0.05% (w/v) to about 0.06% (w/v), 0.06% (w/v) to about 0.07% (w/v), 0.07% (w/v) to about 0.08% (w/v), 0.08% (w/v) to about 0.09% (w/v), 0.09% (w/v) to about 0.10% (w/v), 0.10% (w/v) to about 0.11% (w/v), 0.11% (w/v) to about 0.12% (w/v), 0.12% (w/v) to about 0.13% (w/v), 0.13% (w/v) to about 0.14% (w/v), 0.14% (w/v) to about 0.15% (w/v), 0.15% (w/v) to about 0.16% (w/v), 0.16% (w/v) to about 0.17% (w/v), 0.17% (w/v) to about 0.18% (w/v), 0.18% (w/v) to about 0.19% (w/v), 0.19% (w/v) to about 0.20% (w/v), 0.20% (w/v) to about 0.21% (w/v), 0.21% (w/v) to about 0.22% (w/v), 0.22% (w/v) to about 0.23% (w/v), 0.23% (w/v) to about 0.24% (w/v), 0.24% (w/v) to about 0.25% (w/v), 0.25% (w/v) to about 0.26% (w/v), 0.26% (w/v) to about 0.27% (w/v), 0.27% (w/v) to about 0.28% (w/v), 0.28% (w/v) to about 0.29% (w/v), or 0.29% (w/v) to about 0.30% (w/v) or any integer therebetween.

The load buffer can comprise about 0.001% (w/v), about 0.0015% (w/v), about 0.002% (w/v), about 0.0025% (w/v), about 0.003% (w/v), about 0.0035% (w/v), about 0.004% (w/v), about 0.0045% (w/v), about 0.005% (w/v), about 0.0055% (w/v), about 0.006% (w/v), about 0.0065% (w/v), about 0.007% (w/v), about 0.0075% (w/v), about 0.008% (w/v), about 0.0085% (w/v), about 0.009% (w/v), or about 0.0095% (w/v) non-ionic surfactant P188. The load buffer can comprise about 0.01% (w/v), about 0.015% (w/v), about 0.02% (w/v), about 0.025% (w/v), about 0.03% (w/v), about 0.035% (w/v), about 0.04% (w/v), about 0.045% (w/v), about 0.05% (w/v), about 0.055% (w/v), about 0.06% (w/v), about 0.065% (w/v), about 0.07% (w/v), about 0.075% (w/v), about 0.08% (w/v), about 0.085% (w/v), about 0.09% (w/v), about 0.095% (w/v), about 0.10% (w/v), about 0.11% (w/v), about 0.12% (w/v), about 0.13% (w/v), about 0.14% (w/v), about 0.15% (w/v), about 0.16% (w/v), about 0.17% (w/v), about 0.18% (w/v), about 0.19% (w/v), about 0.2% (w/v), about 0.21% (w/v), about 0.22% (w/v), about 0.23% (w/v), about 0.24% (w/v), about 0.25% (w/v), about 0.26% (w/v), about 0.27% (w/v), about 0.28% (w/v), about 0.29% (w/v), or about 0.3% (w/v) non-ionic surfactant, such as poloxamers (e.g., poloxamer 188 (P188)). The load buffer can comprise 0.20% w/v±0.01% w/v non-ionic surfactant P188. The load buffer of the present disclosure can comprises about 0.001% (w/v), about 0.0015% (w/v), about 0.002% (w/v), about 0.0025% (w/v), about 0.003% (w/v), about 0.0035% (w/v), about 0.004% (w/v), about 0.0045% (w/v), about 0.005% (w/v), about 0.0055% (w/v), about 0.006% (w/v), about 0.0065% (w/v), about 0.007% (w/v), about 0.0075% (w/v), about 0.008% (w/v), about 0.0085% (w/v), about 0.009% (w/v), about 0.0095% (w/v), or about 0.01% (w/v) poloxamer 188. The load buffers of the present disclosure can comprise about 0.005% w/v±0.001% w/v polysorbate 80 or about 0.005% w/v±0.001% w/v poloxamer 188.

### Characterization of AAV Capsids

Several techniques were used for the characterization of AAV capsids. Anion-exchange chromatography (AEX) is a robust tool for the analysis and purification of AAV capsids. AEX allows for the chromatographic separation of full and empty AAVs based upon the surface charge of the capsid, with full capsids exhibiting a different surface charge compared to empty AAVs. Analytical ultracentrifugation (AUC) is another widely used tool for the analysis of AAVs. AUC can provide data on the ratios of empty, partial, and full AAVs within a sample. This process involves centrifuging the sample for several hours, during which the heavier or denser molecules sediment at a faster rate. Mass photometry (MP) has emerged as a powerful technique in recent years for the characterization of AAV capsids. MP permits not only label-free detection and imaging of single molecules, but also mass measurements of individual molecules at elevated levels of accuracy, precision, and resolution. Mass photometry relies on measuring changes in the reflectivity of a glass-water interface caused by interference between scattered by a molecule binding to the interface and light reflected by that interface. For each molecule, the change in the reflectivity produces a mass photometry contrast, which is proportional to the molecular mass of the molecule. MP can provide data on the distribution of masses of species, including the ratios of empty, partial, and full AAVs, by measuring the differences in the interference between the light scattered by a molecule and the light reflected by the surface. MP is a lower resolution technique than both AEX and AUC. Due to the detection limit, mass photometry is suitable for measuring smaller particles, such as proteins, nucleic acids, and small viruses. However, MP is useful for the analysis of AAVs due to its lower sample requirements and high throughput.

### Anion-Exchange Chromatography (AEX)

The following protocol is exemplary and not limiting. Anion exchange ultra-performance liquid chromatography (Waters ACQUITY UPLC H-Class system) can be performed using, for example, a Thermo ProPac SAX-10 column (10 μm, 2 mm×250 mm) (Thermo Fisher Scientific). The column temperature can be kept at an ambient range of about room temperature (about 20° C.-25° C.). The AEX data can be measured and recorded using Fluorescence detectors (FLD), which typically operate at an excitation wavelength of 280 nm and an emission wavelength of 350 nm. All sample vials can be stored in the instrument's auto-sampler, maintained at temperature of about 5° C. An amount, such as 2 μL, of each sample can be injected for analysis without any pretreatment.

For example, mobile phase A can be comprised of 20 mM BTP in Milli-Q water and mobile phase B contained 20 mM BTP and 1 M TMAC in Milli-Q water. Both mobile phases can be prepared using a 0.2 M BTP stock buffer solution with a pH of 9.0. The pH can be then adjusted to 9.5 using sodium hydroxide. The flow rate for AEX can be set at 0.4 mL/min. Initially, the system can be equilibrated with 90% mobile phase A and 10% mobile phase B. The gradient can be then shifted from 90% to 70% for mobile phase A from 0 to 10 minutes. Following this, from 10 to 10.1 minutes, the mobile phase A can decrease from 70% to 10% and this composition can be sustained until 12 minutes. The gradient can be back to 90% mobile phase A and 10% mobile phase B, a ratio that can be maintained until the end of 20-minute running time.

### Analytical Ultracentrifugation (AUC)

The following protocol is exemplary and not limiting. AAV samples can be diluted in, for example, 0.2 μm-filtered placebo buffer, and subsequently loaded (e.g., 100 μL) into the sample chamber of a 2-channel charcoal-epon centerpiece with a 3 mm optical path length, for example. The placebo buffer can be loaded into the reference channel of each cell and subsequently loaded into an eight-cell An50 Ti rotor in an Optima ultracentrifuge (Beckman Coulter, Inc.), for example. An initial 230 nm absorbance scan at about 3,000 rpm preferably can be performed to ensure cell integrity and radial calibration of the optical components. Following the initial scan, the rotor can be allowed to equilibrate to about 20° C. in a vacuum for about 2 hours at rest. For testing, the rotor can be brought to about 25,000 rpm at 20° C., for example. For instance, absorbance scans at 230 nm can be collected every 140 seconds until 100 scans can be collected for each cell. Preferably, the first 40 absorbance scans of the AAV samples can be analyzed using the program SEDFIT (National Institutes of Health) with a continuous c(s) distribution model, determined from numerical solution of the Lamm equation, which describes the time and radial position dependence sedimentation and diffusion in a sector-shaped sample cell as a function of angular velocity. The resulting sedimentation coefficient distribution profiles can be used to calculate the relative abundance of resolved species in solution.

### Mass Photometry (MP)

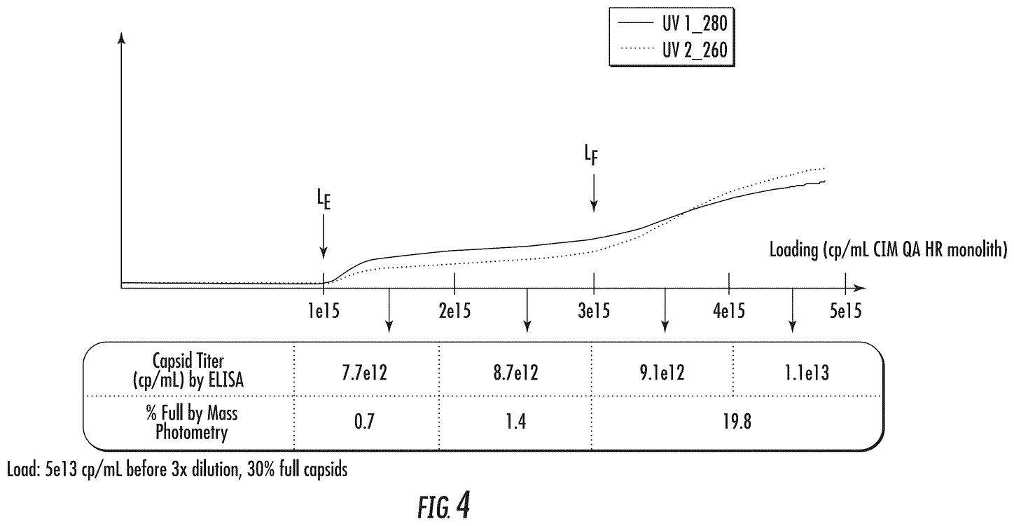

The following protocol is exemplary and not limiting. Mass photometry can be performed using the SamuxMP Auto (Refeyn, Oxford, UK), for example. Experiments can be conducted using the glass cover slips and sample well cassettes provided in the MP Starter kit (Refeyn, Oxford, UK). An amount, for example, 10 μl, of diluted sample can be used for each measurement. Each measurement can be acquired for about 1 minute using the AcquireMP software, for example. Mass photometry measurements can be performed in triplicate. (See for example, FIGS. 4, 6 and 7B).

### EXAMPLES

The inventions are further described by the following Examples, which do not limit the inventions in any manner and are applicable to all sections of the descriptions of the inventions and the aspects of the inventions. The order of performance of the below Examples can be altered or combined as determined by the person of skill in the art in view of the teachings and data contained herein.

The following examples provide teachings on how to purify full rAAV of all serotypes. Polynucleotide and amino acid sequences are widely available in the published literature.

### Example 1: Partitioning AEX Separation of Full-Empty AAV

### Materials and Conditions

Load material—rAAV8; Load % full—30%; Load conductivity—about 6 mS/cm; Chromatographic unit—0.2 mL CIM QA HR SpeCIMen of 800 mL monolith; Buffer A (load buffer)—20 mM bis-tris propane (BTP), 2 mM MgCl₂, 0.001% P188, at pH 9.0; and Buffer B (elution buffer)—Buffer A+250 mM NaCl were used for this experiment.

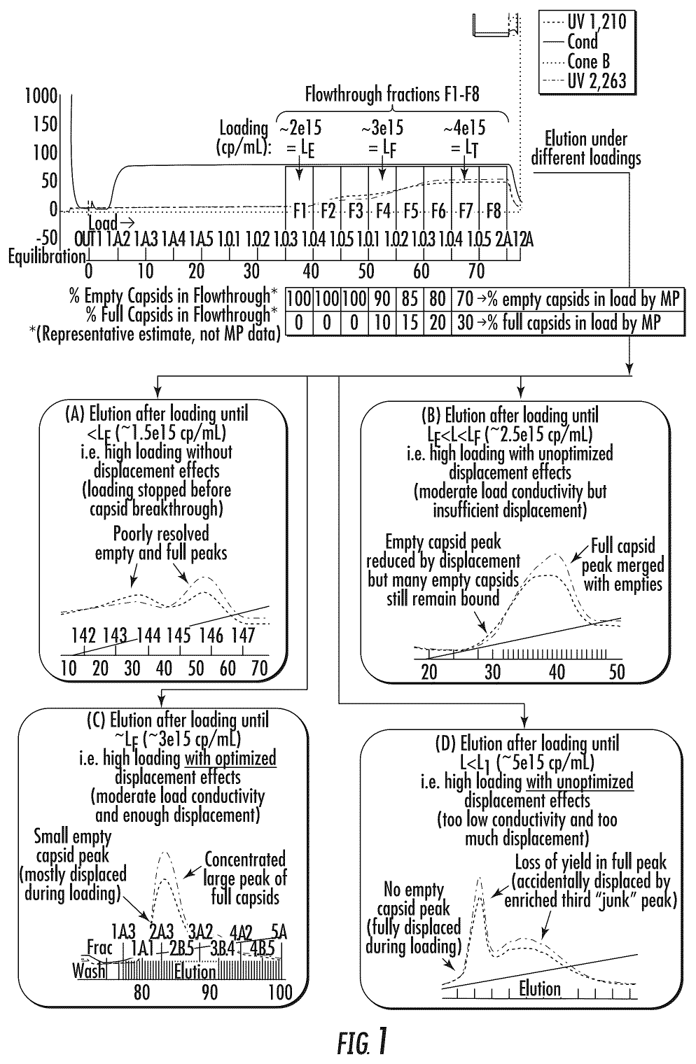

The experiment using serotype AAV8 on CIM QA HR monolith, about 2×10¹⁵ cp/mL loading leads to initial breakthrough of empty capsids (L_{E}). Further loading till about 3×10¹⁵ cp/mL leads to initial breakthrough of full capsids (L_{F}). Yet further loading till about 4×10¹⁵ cp/mL leads to full/empty ratio in the flowthrough approaching that of the load (L_{T}). (see FIG. 1).

Thus, loading up to about 3×10¹⁵ cp/mL can allows most of the binding sites to be taken up by full capsids for the load material studied in this example. After this, a microstep or gradient elution can remove any remaining not full capsids before eluting a peak of full capsids with high concentration, purity, and yield. Optimization of loading amount and load conductivity were critical in this context.

For some serotypes, such as AAV1 and AAV9, some empty capsids can bind more tightly than full capsids, thus displacement separates full capsids in the flowthrough while retaining empty capsids on the column. Two approaches can be used with AAV1 and AAV9:

Option 1 (single column process): Optimize loading amount and load conductivity to get sufficiently enriched full capsids in the flow through.

Option 2 (dual column process): Use two columns in series for loading, with the first column acting as a “sponge” for empty capsids. Load until L>=L_{T} of the first column, capturing all flowthrough material on the second column (this should be enriched in full capsids along with some empty capsids). Then, remove or bypass the first column, and do a microstep elution on the second column.

### Rationale

Higher upstream titers make it increasingly difficult to operate AEX with loading in the order of about 1×10¹⁴ cp/mL as in current processes, but higher loadings have been shown to lower % full in the AEX pool.

For example, loading at 1×10¹⁴ cp/mL requires 2 cycles on an 8000 mL monolith at 500 L bioreactor scale with an upstream titer of 1×10¹² vg/mL and 3×10¹² cp/mL (total 1.5×10¹⁸ cp). Monolith cycling is a challenge leading to higher costs. Also, large monoliths and multiple cycles increase processing time and result in high volumes (>50L) of AEX pool which are difficult to process with Viral retentive filtration (VRF) and Ultrafiltration and diafiltration (UFDF). Large pool volumes also lower the AEX pool concentration, impacting stability as observed in previous studies due to reduced capsid self-buffering effects.

In contrast, partitioning chromatography allows for >20-fold higher loading at about 2 to 4×10¹⁵ cp/mL, thus one cycle on a 400 mL monolith would suffice for a 500 L batch. This is a >20-fold reduction in monolith volume requirements and cost. This is also a >20-fold reduction in pool volume corresponding to an increase in pool concentration improving stability via increased capsid self-buffering.

Key factors affecting the performance include:
Loading (cp/mL);
Load buffer conductivity (2-12 mS/cm);
Percent (%) Full/Empty capsids ratio in load;
AAV serotype (empty binds weaker than full, for example, AAV2, AAV8; or empty binds tighter than full, for example, AAV1, AAV9, AAV9-SpyT);
Load buffer pH (6.0-11.0); and
Load buffer composition (with or without MgCl₂, NaCl/NaAc salt, pH 7.5-11.0).

### Example 2—Rapid-Cycling Chromatography for AAV Process Intensification

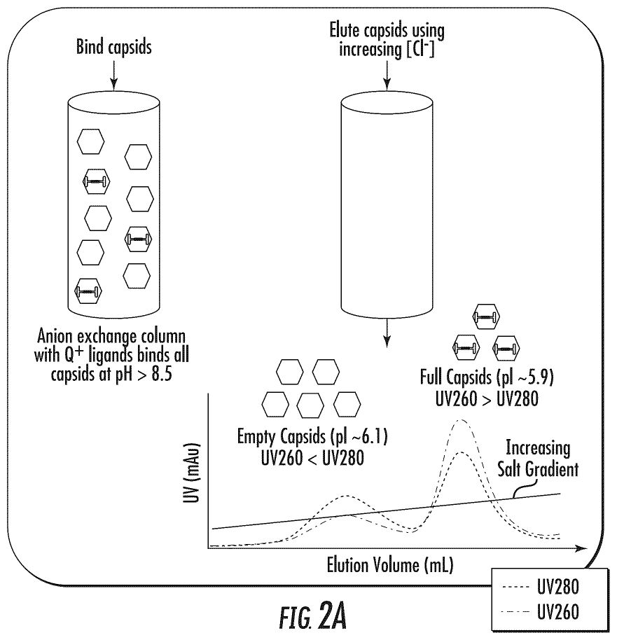

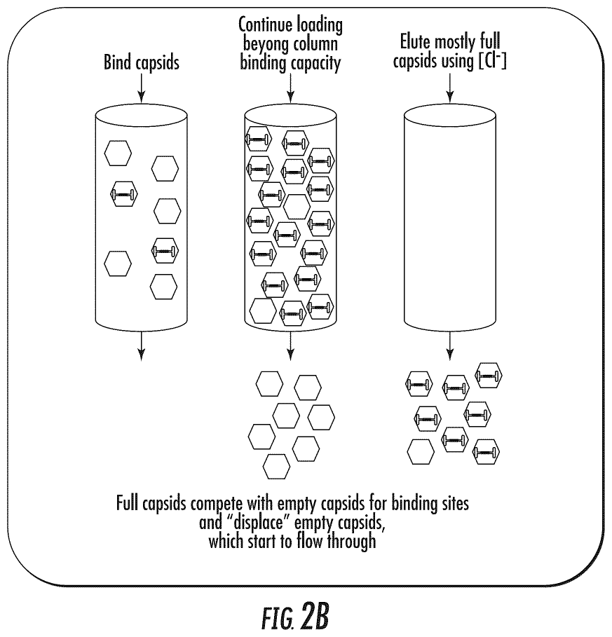

FIGS. 2A and 2B depict that partitioning AEX (FIG. 2B) is an alternative to standard bind-and-elute AEX (FIG. 2A), which is traditionally conducted in linear gradient mode. The partitioning AEX is suitable for continuous processing as it allows for isocratic elution and rapid cycling.

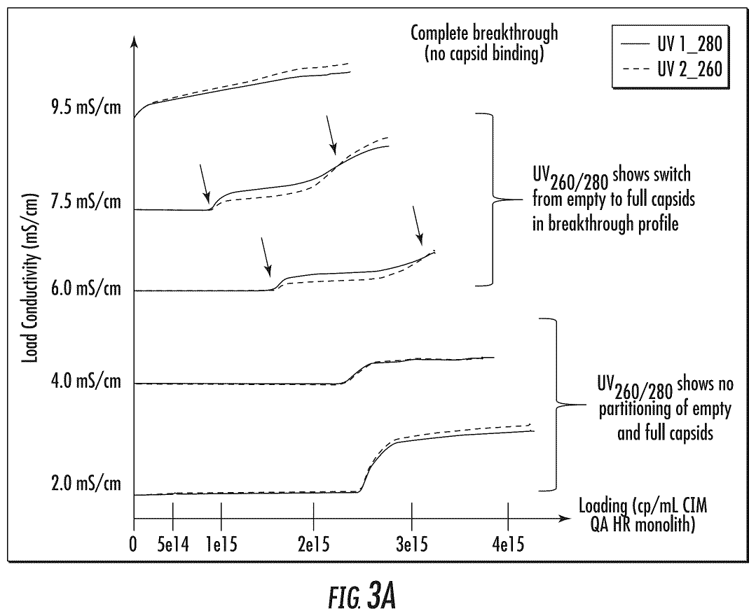

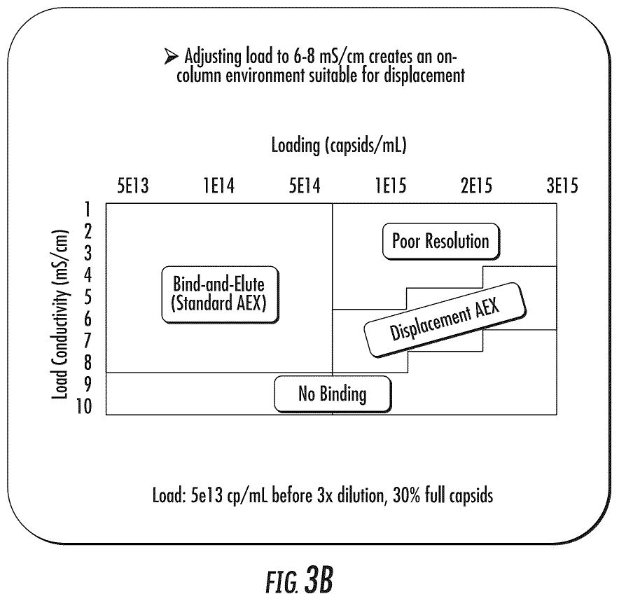

FIGS. 3A and 3B illustrate that the loading amount and conductivity are critical parameters impacting whether capsid displacement occurs. UV_{260/280} shows switch from empty to full capsids in breakthrough profile (FIG. 3A). Adjusting load to 6-8 mS/cm creates an on-column environment suitable for displacement (FIG. 3B). Additionally, varying the amount of MgCl₂ in the load material and buffer systems can modify the displacement effects achieved.

FIG. 4 shows capsid titer and mass photometry (MP) analysis of breakthrough fractions and confirms displacement effect observed using UV. Ideal loading window appear to be after empty capsids begin to breakthrough (“L_{E}”) until before full capsids begin to breakthrough (“L_{F}”).

FIG. 5 depict linear gradient elutions graphs and data support that loading to achieve enough displacement is used for the separation of empty and full capsids. The graphs illustrate loading within different displacement zones, followed by linear gradient elution using increasing NaCl.

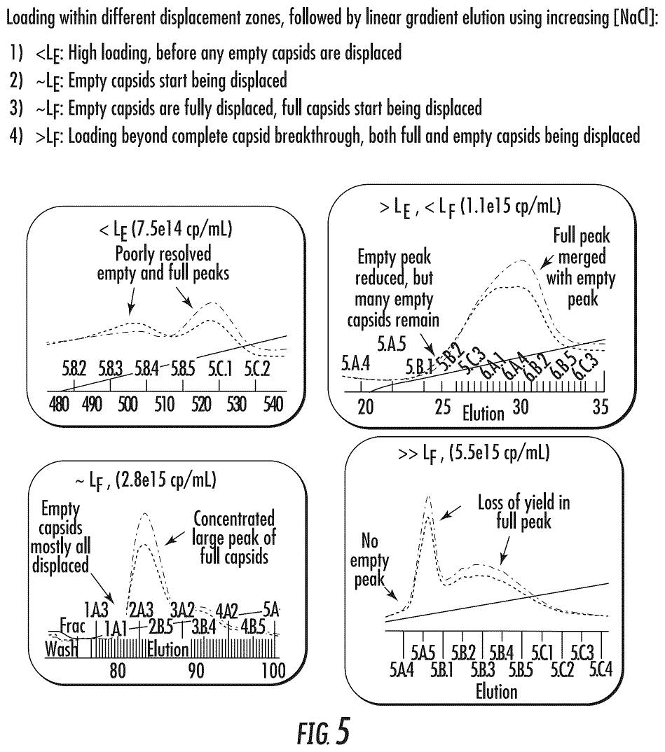

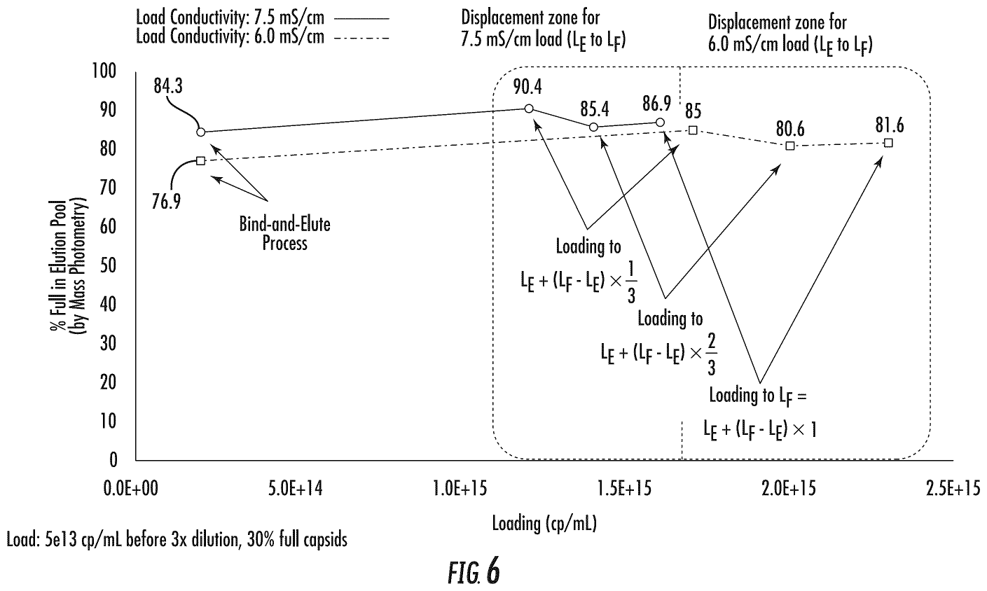

FIG. 6 showing improved % full capsids compared to bind-and-elute process for loadings inside the displacement zone.

Real-time approach for loading cutoff allows for rapid cycling with high yield and purity using displacement chromatography. Tracking UV_{260/280} ratio of capsid breakthrough profile shows consistency between units and across scales (FIG. 7A). FIG. 7B depicts Mass Photometry (MP) histogram of elution pool showing 95.7% full capsids by MP.

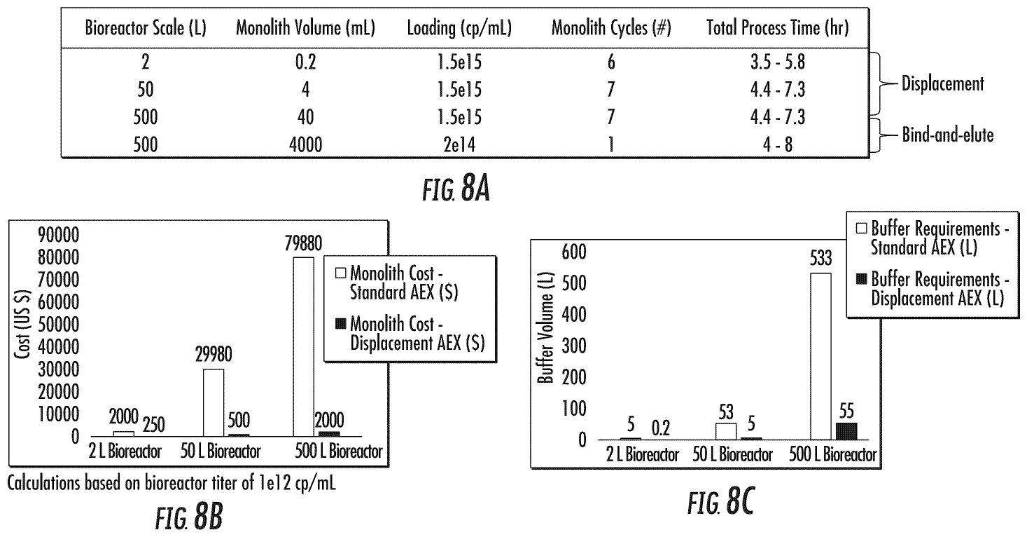

FIGS. 8A-8C showing that rapid cycling displacement chromatography lowers monolith volume by about 100-fold (FIG. 8A); estimated associated cost by about 40-fold for 500L bioreactor, about 60-fold for a 50L bioreactor and about 8-fold for 2L reactor (FIG. 8B); and buffer volume by about 10-fold (FIG. 8C). Total processing time for the bind-and-elute experiment was between about 4 to 8 hours. Total processing time for the rapid cycling displacement chromatography experiments were between about 3.5 to 7.3 hours. Use of smaller units allows for 10 cycles to be conducted instead of 1 cycle with high flow rates of 3-5 CV/min, compared to 0.5-1 CV/min for 4-8 L units due to pressure limitations.

FIG. 9 schematically depicts that continuous processing principles can be used to intensify AAV purification processes. Key benefits include 10× smaller affinity column, 100× smaller monolith, and 10× lower buffer requirements. The rapid cycling displacement chromatography process also is suitable for continuously managing multiple upstream batches staggered by 2 days of harvest time at 500 L scale.

### Example 3—Downstream Manufacturing of AAV using Continuous Processing Principles—An Overview of AAV Downstream Purification Process

FIG. 10 depicts exemplary production purification trains for AAV, such as recombinant AAV. The top train uses a batch process where repeated passes are required to exchange buffer and concentrate the retentate, which contains the desired biological material, such as AAV. See Adams et al., Biotech. Bioeng. 117:3199-3211 (2020).

The bottom section of FIG. 10 replaces the batch tangential flow filtration unit with a single-pass tangential flow filtration unit (SPTFF unit), which permits a continuous process. It was surprising how well SPTFF performed with AAV, as disclosed herein.

The Batch TFF approach can take multiple days (for example, four days) due to the repeated cycling through the conventional TFF unit to achieve concentration prior to further purification. The SPTFF approach is a continuous approach, and is significantly faster than the Batch TFF approach, and can be performed in several hours, such as 3 to 5 hours. The SPTFF approach provides faster concentration, while minimizing sheer stress and damage to AAVs. The SPTFF approach also is amenable to the use of Process Analytical Technology (PAT) and automation. The current inventions provide continuous processing techniques including Single-Pass TFF (SPTFF) and multi-column or rapid-cycling chromatography (FIG. 10).

### Example 4-Partitioning AEX for AAV: L_{E}, L_{F} and k_{WP}

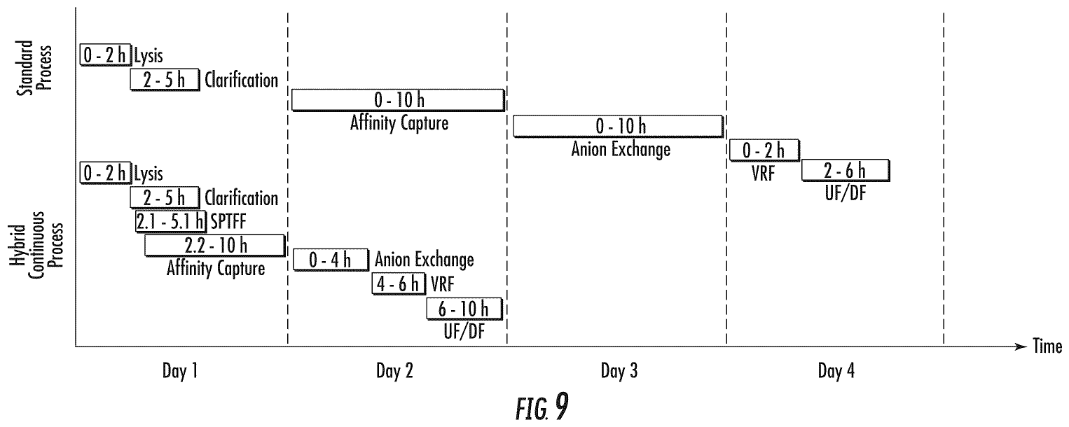

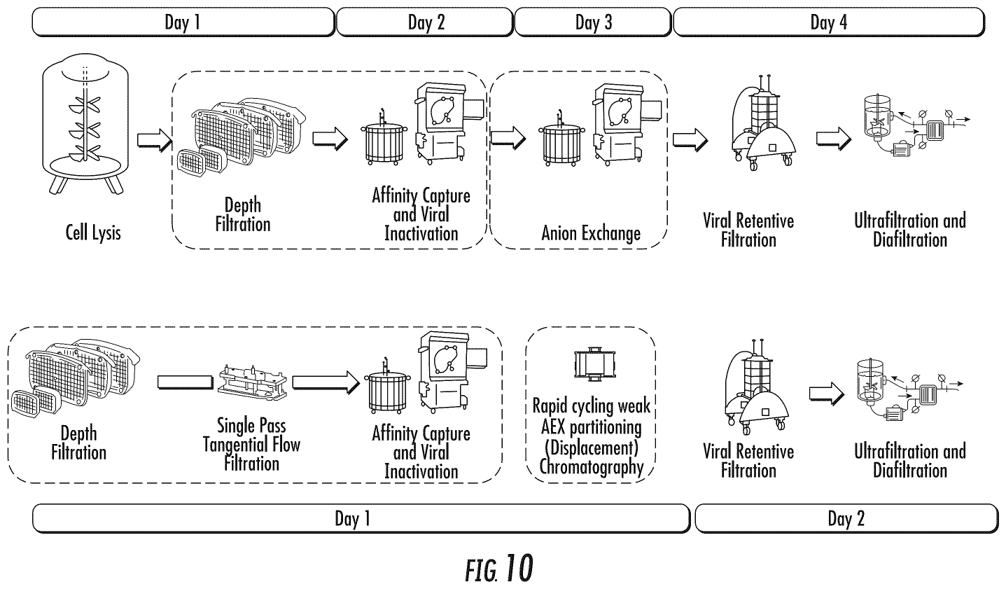

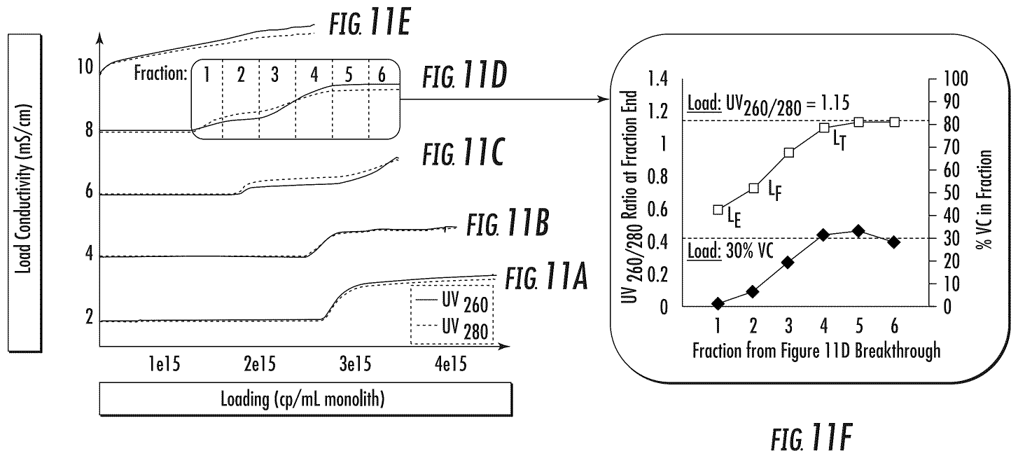

To better describe the loading amounts used with reference to weak partitioning AEX, the following nomenclature was adopted: L_{E} is the capsid loading required to begin breakthrough of EC species, and L_{F} is the loading required to begin breakthrough of VC species. For example, L_{E}<L_{F} when weak partitioning occurs (FIGS. 11C and 11D), while L_{E}≈L_{F} for the runs where weak partitioning does not occur (FIGS. 11A, 11B and 11E). Loading until L_{F} is optimal as this results in almost complete displacement of EC into the flowthrough without loss of VC.

Since mass photometry is an offline and time-consuming measurement, it is not feasible to conduct this analysis on all breakthrough samples. As an alternative, the UV₂₆₀ and UV₂₈₀ signals can be used to rapidly identify L_{E} and L_{F}, as can be seen in FIG. 11F. L_{E} was defined as the loading amount where the UV₂₈₀ signal began to increase from baseline and is also a measure of dynamic binding capacity at 0% breakthrough for the monolith under the loading condition being tested. L_{F} was defined as the loading amount where UV_{260/280}=0.7, representing the beginning of VC breakthrough as can be seen in FIG. 11F.

Finally, a weak partitioning coefficient (k_{WP}) was defined as given in Equation 1.

$${k}_{WP} = \frac{{L}_{F} - {L}_{E}}{{L}_{F}} \qquad ( Equation 1 )$$

The coefficient measures the fraction of total loaded capsids (L_{F}) that are being leveraged to displace EC, as the L_{F}-L_{E} difference corresponds to the amount of EC that are being displaced into the flowthrough. A higher value of k_{WP} indicates that a larger fraction of the total loaded capsids prior to VC breakthrough are being leveraged for weak partitioning. Thus, higher k_{WP} is expected to correspond to improved VC enrichment. This parameter is useful in comparing weak partitioning effects across runs with different load conditions, and to use as a quantitative maximization target. Note that k_{WP} is expected to be independent of capsid concentration, as a more dilute load would increase the values of L_{E} and L_{F} proportionally.

FIGS. 11A to 11F showing the results of monitoring AEX flowthrough using UV₂₆₀ and UV₂₈₀ signals during overloading of AAV8 capsids on CIM QA monolith at load conductivities of 2 (FIG. 11A), 4 (FIG. 11B), 6 (FIG. 11C), 8 (FIG. 11D), and 10 (FIG. 11E) mS/cm, showing weak partitioning effects for FIG. 11C and FIG. 11D. FIG. 11F is an offline mass photometry analysis of flowthrough fractions from FIG. 11D, showing EC breakthrough followed by VC breakthrough, confirming weak partitioning effect observed using UV₂₆₀ and UV₂₈₀ signals.

### Example 5—Variation in load % VC, pH, and additive concentration on weak partitioning AEX

Previous experiments showed the importance of maintaining load conductivity in the range of 6-8 mS/cm to allows for weak partitioning. Further avenues for characterization and optimization of weak partitioning AEX include varying other load parameters such as load pH and concentration of Mg²⁺, which are known to influence the binding strength and surface charge characteristics of AAV capsids. Modulation of load pH in the range of 6.5-10.5, as well as [Mg²⁺] in the range of 0-6 mM has been reported to affect resolution between EC and VC, as has the presence of other ions such as sulfate or ammonium. The starting % VC from upstream as well as differences in upstream material characteristics are also well-known to impact the final pool purity achievable using a diverse range of AEX methods.

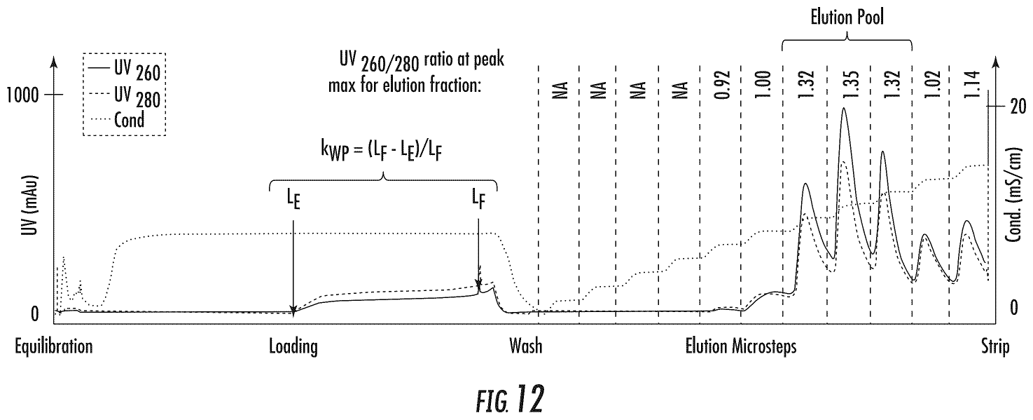

To further investigate load characteristics, a set of 32 runs were conducted as shown in Table 1. Elutions were conducted using microsteps of 1 mS/cm ranging from 2 to 12 mS/cm. For these experiments, microsteps were used rather than linear gradients to provide a standard approach for automated fractionation and post-run pooling decisions. All fractions with UV_{260/280} ratio at peak maximum >1.20 were pooled and analyzed for % VC and genomic yield, as summarized in Table 1. Lastly, the values of L_{E}, L_{F} and k_{WP} were manually calculated for each run based on the UV₂₆₀ and UV₂₈₀ signals in the load flowthrough. An example chromatogram showing Run 8 in Table 1 also is shown in FIG. 12, along with annotations to show key loading and pooling parameters.

TABLE 1

| | | | | | | | |
|---|---|---|---|---|---|---|---|
| | | | | | | | Elution |
| | | Load | | | | Elution | Pool % |
| Run | Load | [MgCl₂] | Load | | | Pool % | Genomic |
| No. | pH | (mM) | % VC | L_{F} | k_{WP} | VC | Yield |

(A) Varying Load pH and [MgCl₂]

| | | | | | | | |
|---|---|---|---|---|---|---|---|
| 1 | 6.5 | 0 | 32 | 5.6e14 | 100.0 | 71.1 | 19.2 |
| 2 | | 2 | | 4.5e14 | 100.0 | 67.5 | 18.0 |
| 3 | | 4 | | 2.2e14 | 100.0 | 59.4 | 29.0 |
| 4 | 7.5 | 0 | | 1.5e15 | 55.6 | 70.7 | 48.3 |
| 5 | | 2 | | 5.4e14 | 58.0 | 78.4 | 38.4 |
| 6 | | 4 | | 5.0e14 | 57.4 | 70.7 | 40.8 |
| 7 | | 6 | | 3.6e14 | 40.2 | 70.1 | 54.3 |
| 8 | 8.5 | 0 | | 2.2e15 | 49.8 | 71.5 | 51.0 |
| 9 | | 2 | | 1.7e15 | 68.9 | 78.6 | 60.3 |
| 10 | | 4 | | 1.2e15 | 66.3 | 80.6 | 63.2 |
| 11 | | 6 | | 6.5e14 | 64.8 | 76.3 | 41.5 |
| 12 | 9.5 | 0 | | 2.6e15 | 27.8 | 66.8 | 43.5 |
| 13 | | 2 | | 2.4e15 | 46.1 | 71.6 | 43.0 |
| 14 | | 4 | | 2.3e15 | 44.9 | 72.7 | 55.3 |
| 15 | | 6 | | 2.0e15 | 43.8 | 66.6 | 43.3 |
| 16 | 10.5 | 0 | | 5.6e14 | 100.0 | 71.1 | 19.2 |
| 17 | | 2 | | 4.5e14 | 100.0 | 67.5 | 18.0 |
| 18 | | 4 | | 2.2e14 | 100.0 | 59.4 | 29.0 |
| 19 | | 6 | | 1.5e15 | 55.6 | 70.7 | 48.3 |

(B) Varying Load % VC

| | | | | | | | |
|---|---|---|---|---|---|---|---|
| 20 | 8.5 | 0 | 15 | 1.2e16 | 75.0 | 53.5 | 36.9 |
| 21 | | 4 | | 5.8e15 | 78.6 | 68.2 | 61.8 |
| 22 | | 6 | | 3.7e15 | 76.3 | 59.1 | 47.6 |
| 23 | | 0 | 65 | 1.6e15 | 39.8 | 90.4 | 71.3 |
| 24 | | 2 | | 1.5e15 | 38.5 | 89.5 | 70.0 |
| 25 | | 4 | | 1.4e15 | 28.9 | 84.9 | 68.0 |
| 26 | | 6 | | 1.2e15 | 26.8 | 83.2 | 69.5 |

(C) Varying Additives

| | | | | | | | |
|---|---|---|---|---|---|---|---|
| 27 | 8.5 | No Additive | 32 | 3.2e15 | 61.9 | 77.7 | 38.3 |
| 28 | | 2 mM MgCl₂ | | 2.5e15 | 74.6 | 81.1 | 55.4 |
| 29 | | 2 mM MgSO₄ | | 2.5e15 | 68.6 | 84.6 | 43.2 |
| 30 | | 2 mM (NH₄)₂SO₄ | | 2.3e15 | 68.9 | 79.6 | 45.3 |
| 31 | | 2 mM MgCl₂ + | | 1.3e15 | 72.3 | 80.2 | 36.6 |
| | | 2 mM MgSO₄ | | | | | |
| 32 | | 2 mM MgCl₂ + | | 1.8e15 | 61.6 | 74.9 | 57.9 |
| | | 2 mM (NH₄)₂SO₄ | | | | | |

FIG. 12 is showing an example of a weak partitioning AEX chromatogram showing calculations of L_{E}, L_{F} and k_{WP} and elution using conductivity increments of 1 mS/cm. All peaks with UV_{260/280}>1.2 were pooled and analyzed for 32 runs listed in Table 1 (Run 8 from Table 1 is shown in FIG. 12 as an example).

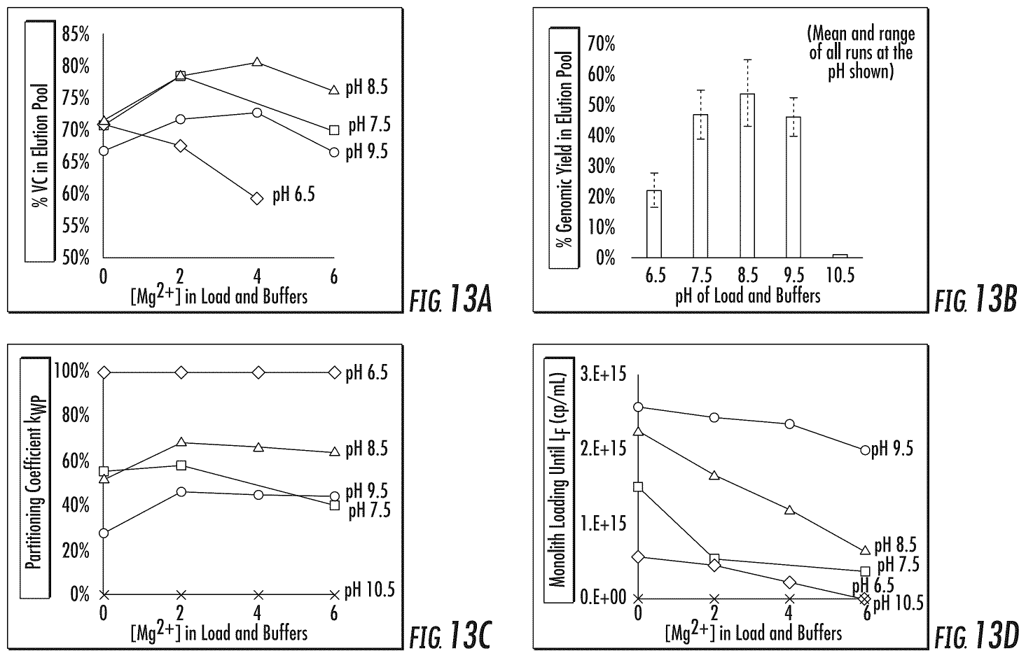

Results from Table 1 (A) are depicted graphically in FIGS. 13A to 13D. The Figures illustrate AEX elution pool % VC (FIG. 13A) and genomic yield (FIG. 13B), along with weak partitioning AEX parameters partitioning coefficient k_{WP} (FIG. 13C) monolith loading until VC breakthrough (FIG. 13D), for AEX runs conducted with different load pH and [Mg²⁺].

Operating at pH 8.5 with 2-4 mM of MgCl₂ resulted in an optimal maximum in terms of % VC as well as k_{WP}, and this pH also resulted in highest genomic yield. Notably, moving 1 pH unit lower or higher to pH 7.5 or 9.5 adversely impacted all three responses of % VC, k_{WP}, and genomic yield.

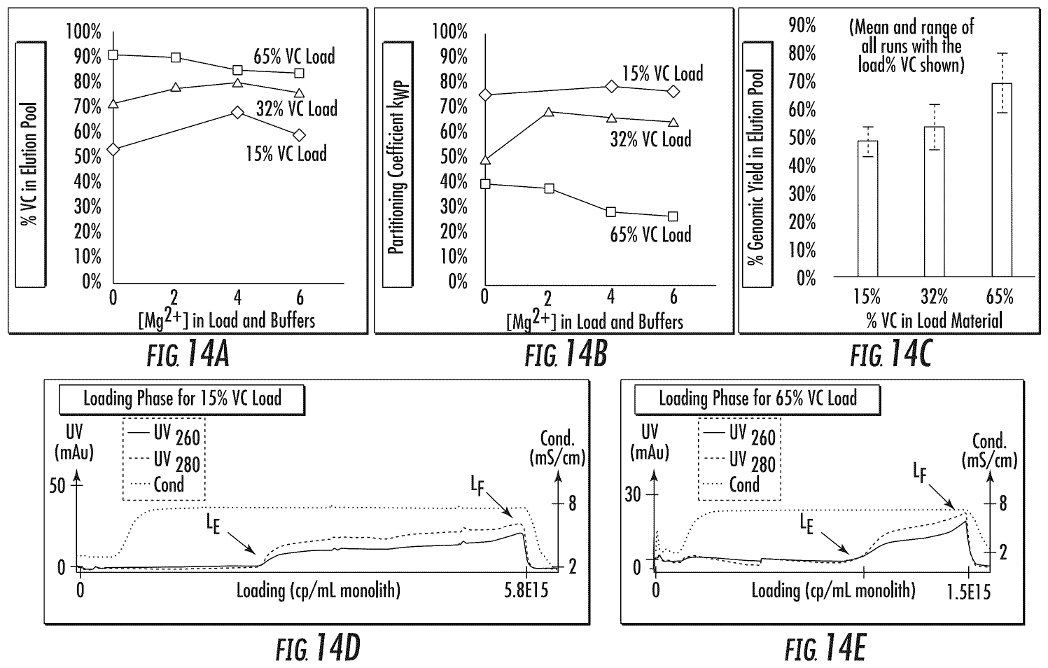

Results from Table 1 (B) are depicted in graphs in FIGS. 14A to 14E. The Figures showing AEX elution pool % VC (FIG. 14A), partitioning coefficient k_{WP} (FIG. 14B), and genomic yield (FIG. 14C) for AEX runs conducted with different starting % VC in the load material at constant pH of 8.5. The impact of starting % VC on k_{WP} is illustrated further through (FIG. 14D) and (FIG. 14E) comparing loads with 8% and 65% VC, respectively.

FIGS. 14A to 14E illustrate the impact of load % VC on the performance of weak partitioning AEX when operated at pH 8.5 with varying [Mg²⁺]. Like the results in the FIGS. 13A-13D, [Mg²⁺] of 2-4 mM appears optimal. The impact of starting % VC on k_{WP} can be observed through these experiments. For example, for starting % VC of 65%, the average value of k_{WP} reached across different [Mg²⁺] conditions was 35%. In contrast, k_{WP} values of 65% could be reached for load material with 32% VC, and k_{WP} values of 80% could be reached for load material with 15% VC. Logically, this trend is expected as it is not possible to displace a higher percentage of EC than has physically been loaded onto the column as a proportion of the total loaded capsids. This is based on binding equilibria between EC and VC, which is determined by the starting % EC/VC in the load material.

FIGS. 14D and 14E further illustrate the impact of starting % VC on k_{WP}, showing the extended period of EC breakthrough observed for load material with 15% VC, compared to a much shorter period of EC breakthrough for load material with 65% VC. It is notable that the measured % k_{WP} value was within 10% of the value of (100%—starting % VC) for all three types of load materials, which were all generated from separate upstream productions with different transgenes and transfection strategies. This supports the hypothesis that the weak partitioning method is not significantly impacted by the nature or size of the therapeutic transgene inside the AAV8 capsid. This also suggests that it is useful to target a value of (100%-starting % VC) as a target % k_{WP} for optimization of weak partitioning AEX.

### Example 6-Developing of Isocratic Elution Strategy for Weak Partitioning AEX Suitable for Large-Scale GMP Manufacturing of AAV8

Studies using an optimized load conductivity (6-8 mS/cm), Mg2+ concentration (2-4 mM), pH (˜8.5), and loading target (˜ LF) for weak partitioning AEX. The combination of these conditions provided successful enrichment of % VC in the AEX pools which were collected through linear gradient or microstep elutions. However, it was possible to eliminate the need for either elution gradients or microsteps by designing a control strategy for the loading and wash phases that ensures removal of EC via weak partitioning effects alone, enabling VC to be eluted in an isocratic step elution. For this approach to be feasible for large-scale GMP production, four criteria were outlined as below. The following set of studies was conducted to develop a strategy for weak partitioning AEX suitable for large-scale GMP manufacturing.
i. The range of allowable conductivities for the elution buffer required was reasonably wide, for example, 2 mS/cm difference between the minimum and maximum allowable conductivities.
ii. The range of bioreactor scales and titers that can be processed was wide enough to encompass both current lower- and higher-end titers as well as projected future bioreactor titers, for example, 50-2000 L scale bioreactors with titers ranging from 1e11 to 1e14 cp/mL upstream.
iii. The operation was not sensitive to changes in the capsid concentration or % VC of the load material in case of variations, dilution, or yield hits in the previous unit operations.
iv. The operation was feasible on existing pilot- and manufacturing-scale chromatography skids without the need for manual intervention or additional pumps, valves, or sensors.
Correlation Between Load and Elution Conductivities for Weak Partitioning AEX:

A feature of weak partitioning AEX is that the load conductivity impacts the elution conductivity, and this effect can be used to develop a robust isocratic elution process that is not sensitive to the exact conductivity of a the high-salt buffer that is used to control the elution. To demonstrate this, five experiments were conducted as shown in Table 2. Load material with 32% VC was adjusted to pH 8.5 and 3 mM MgCl₂ to match the optimized condition for weak partitioning developed in Example 6, and the conductivity was adjusted to 6 or 8 mS/cm. Loading was performed up until L_{F} and L_{E}+0.66×(L_{F}−L_{E}), corresponding to loading until VC breakthrough as well as ˜33% lower within the weak partitioning zone. An additional control run was conducted with loading to 2e14 cp/mL («L_{E}) at a load conductivity of 2 mS/cm to match the conditions used for linear gradient AEX. Loading value of 2e14 cp/mL is referred as «L_{E}. All elution fractions with UV_{260/280} >1.2 at peak maximum were pooled from each run, and the conductivities of the pooled peaks and % VC were measured.

TABLE 2
Experiments investigating the impact of load conductivity on elution conductivity in standard
linear gradient AEX (Runs 1 and 2) compared to weak partitioning AEX (Runs 3-6).

| | | | | Starting | | |
|---|---|---|---|---|---|---|
| | | | | conductivity | | |
| | | | | of elution | | % genomic |
| | | Loading | Load | fractions with | % VC in | yield in |
| Run | Loading relative | (cp/mL | conductivity | UV_{260/280} > 1.2 | elution | elution |
| No. | to L_{E} and L_{F} | monolith) | (mS/cm) | (mS/cm) | pool | pool |
| 1 | «L_{E} | 2.0e14 | 2 | 10 | 76 | 49 |
| 2 | L_{E} + 0.66x(L_{F} − L_{E}) | 2.7e15 | 6 | 7 | 81 | 61 |
| 3 | L_{F} | 2.9e15 | | 7 | 82 | 42 |
| 4 | L_{E} + 0.66x(L_{F} − L_{E}) | 1.8e15 | 8 | 9 | 85 | 56 |
| 5 | L_{F} | 2.0e15 | | 9 | 87 | 56 |

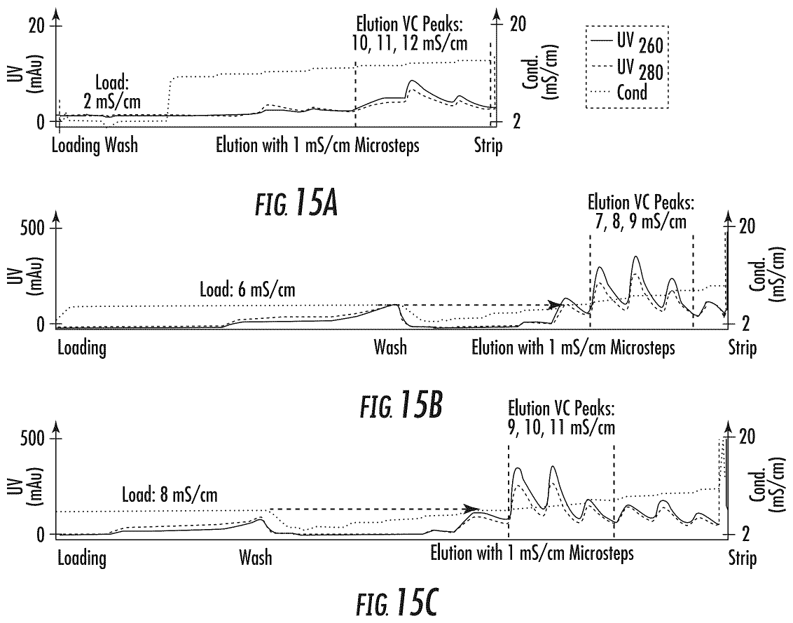

These experiments demonstrated that when weak partitioning AEX is used, the VC peaks began to elute at a fixed delta of 1 mS/cm higher than the load conductivity, that is, at 7 mS/cm when the load conductivity was 6 mS/cm, or at 9 mS/cm when the load conductivity was 8 mS/cm (Runs 3 and 5). This can be seen in FIGS. 15B-15C, where the elution conductivities of the VC peaks with UV_{260/280}>1.2 are annotated above the chromatograms. Runs 2 and 4, which were not loaded until L_{F}, also exhibited this trend, suggesting that the loading exactly until VC breakthrough was not critical to observe this effect.

This contrasts with the control run without weak partitioning, where the VC peaks began to elute at 10 mS/cm (Run 1, FIG. 15A). Note that when loading to «L_{E}, elevating the load conductivity to 6 or 8 mS/cm still resulted in elution of the VC peaks at 10 mS/cm. The hypothesis is that the saturation of binding sites along with elevated conductivity reduces the number of quaternary ammonium (Q+) ligands bound to an individual AAV capsid when operating in weak partitioning mode. This weak binding is not only what allows for displacement of EC by VC during loading, but also results in a lower conductivity barrier to overcome for elution. Thus, even a slight increase in conductivity is sufficient to elute the bound VC. The dependence of elution conductivity on load conductivity, as well as the wide range of acceptable load conductivity between 6 and 8 mS/cm, creates a situation where the high-salt buffer conductivity can be prepared against a wider specification than is typically possible for isocratic elutions. The need for narrow conductivity specifications has been one of the factors limiting successful scale-up of isocratic processes for EC/VC separations in the industry due to impacts of slight variations in buffer preparation methods or operating room temperature on conductivity measurements. For example, if seeking to convert the AEX process in FIG. 15A to an isocratic elution, one pre-elution step would be needed at 9±0.1 mS/cm to remove EC, followed by another at 12±0.1 mS/cm to elute VC. This requires the buffers to be prepared very precisely, and even with the use of a gradient pump, at least one buffer must be made to the narrow specification.

In contrast, to convert the weak partitioning AEX process in FIGS. 15B-15C to an isocratic elution, the high-salt buffer can be prepared with conductivity in the range of about 9 to 11 mS/cm. Leveraging a gradient pump for loading with 66% elution buffer and 33% load material results in a 3× dilution along with adjustment of a about 2 mS/cm load to between 6 and 8 mS/cm, which is ideal for weak partitioning AEX. This can be followed by an elution with 100% B corresponding to a conductivity of about 9 to 11 mS/cm which is ideal for elution and prevents collection of the late impurity peaks that elute at higher conductivities than the main VC peaks. In this way, a single buffer is used to control both the load and elution conductivities, resulting in a process that can withstand variations between 9 and 11 mS/cm for the high salt buffer. This is an acceptance range of 2 mS/cm and is several-fold wider than the range required for buffers generally needed for isocratic processes.

An effective way to execute this process is illustrated in FIG. 16. An equilibration buffer with a similar conductivity as the undiluted AEX load material (about 2.0 mS/cm) is prepared and diluted in-line using a gradient pump with elution buffer in the same manner as the load material, to achieve similar conductivity across equilibration, loading and wash phases. Utilizing in-line dilution for pre-load, load and post-load steps helps to guard against conductivity spikes that may arise when changing gradient percentages at large scale. The approach also ensures that any remaining load material in Pump A will be appropriately diluted and pushed onto the AEX monolith by the wash buffer at the start of the wash phase, reducing yield loss. To end the wash phase, either a CV-based or UV_{260/280} ratio-based signal can be used, similar to the trigger used to end the load phase.

### Example 7—Operating Weak Partitioning AEX for Large-Scale Manufacturing

FIG. 16 provides a schematic of strategies for weak partitioning AEX with isocratic elution, using a single high-salt elution buffer with gradient pump to control both load dilution as well as elution, resulting in a process suitable for large-scale operations that is robust within conductivity variations of 9-11 mS/cm for the buffer.

An effective way to execute this process is illustrated in the FIG. 16. An equilibration buffer with a similar conductivity as the undiluted AEX load material (about 2.0 mS/cm) is prepared and diluted in-line using a gradient pump with elution buffer in the same manner as the load material, to achieve similar conductivity across equilibration, loading and wash phases. Utilizing in-line dilution for pre-load, load and post-load steps helps to guard against conductivity spikes that may arise when changing gradient percentages at large scale. The approach also ensures that any remaining load material in Pump A will be appropriately diluted and pushed onto the AEX monolith by the wash buffer at the start of the wash phase, reducing yield loss. To end the wash phase, either a CV-based or UV_{260/280} ratio-based trigger can be used, like the trigger used to end the load phase.

### Example 8-Isocratic elution using weak partitioning AEX

Optimization of the wash phase between loading and elution is a key element of developing a manufacturing-ready method. This is because even though most of the bound EC have been displaced by VC at the end of the loading phase, a proportion of the displaced EC population remains within the system hold-up volume. Thus, a buffer wash through the system is needed to remove this material and prevent it from mixing with the target isocratic elution pool. By maintaining pH and conductivity of the wash buffer equivalent to that of the load material, the wash can prevent binding and continue to conduct displacement of any unbound EC in the pre-monolith system hold-up volume as well as in the monolith itself.

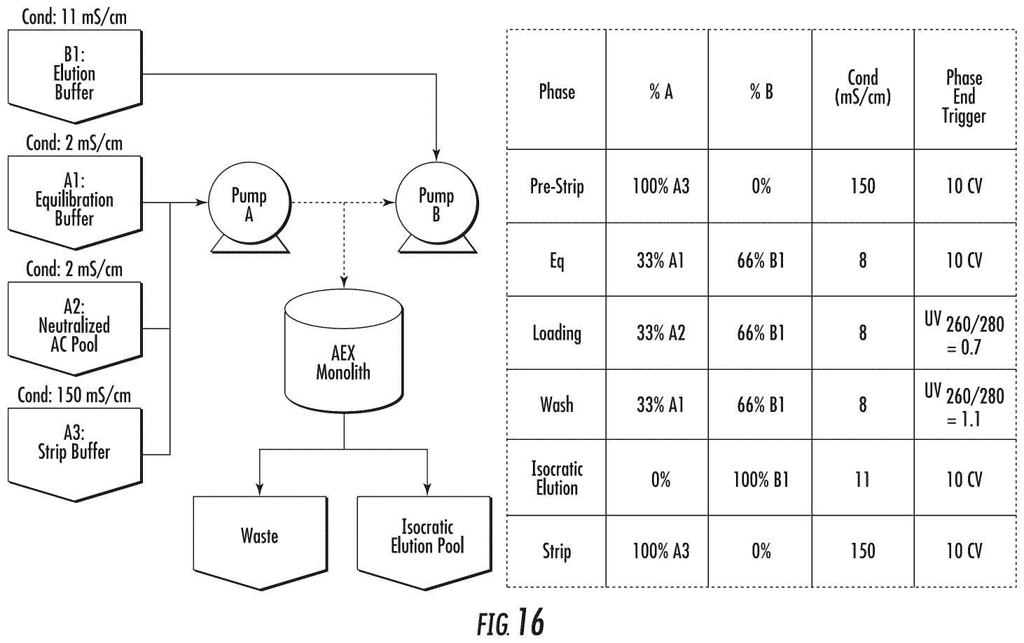

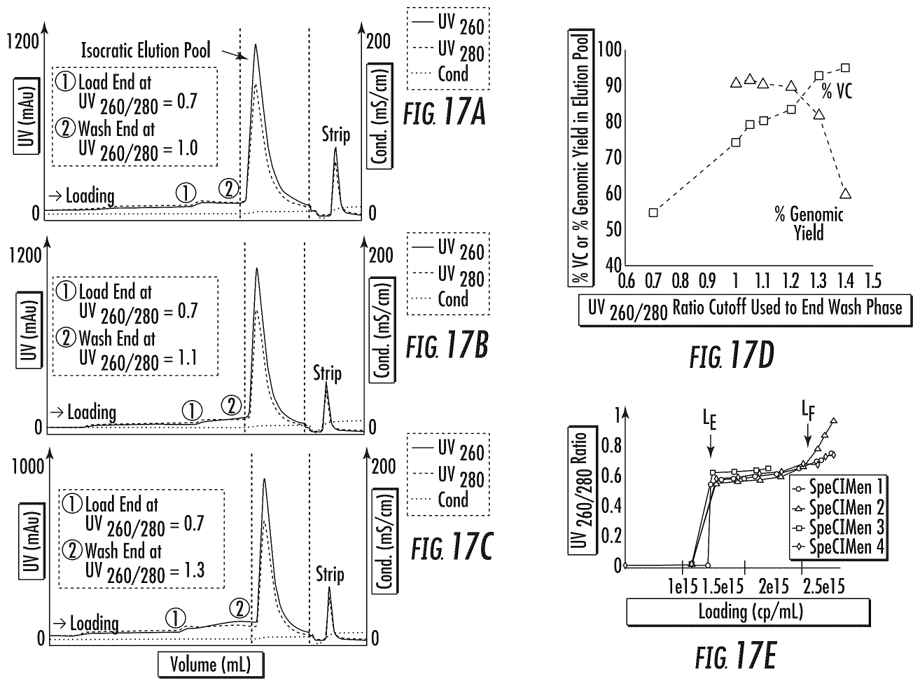

Runs (N=7) were executed to evaluate the manufacturing strategy, and the results of the test runs are summarized in FIGS. 17A to 17E. The UV_{260/280} ratio at the monolith outlet was monitored during loading until a value of 0.7 indicating L_{E} was reached, which triggered the end of the load phase and the start of the wash phase. The UV_{260/280} ratio at the monolith outlet was again monitored until target cut-offs of 0.70, 1.00, 1.05, 1.10, 1.20, 1.30 and 1.35 were reached (N=7 independent runs), which triggered a switch to 100% (FIG. 17B) for the isocratic elution. The load phase resulted in similar UV_{260/280} profiles in the flow through, after which both UV signals displayed a slight increase indicating the start of the wash phase. This was allowed to continue for different durations depending on the UV_{260/280} trigger used to end the phase, after which the isocratic elution was collected. Chromatograms from runs with UV_{260/280} cutoff of 1.00, 1.10 and 1.30 to end the wash phase are shown in FIGS. 17A-17C. The Figures illustrate chromatograms of weak partitioning AEX with isocratic elution using UV_{260/280} ratio triggers of (FIG. 17A) 1.0, (FIG. 17B) 1.1 and (FIG. 17C) 1.3 to end the wash phase; (FIG. 17D) Impact on % VC and % genomic yield in the isocratic elution pool; (FIG. 17E) Comparable UV_{260/280} ratio profiles during weak partitioning load for four different CIM QA SpeCIMen units sourced from parent monoliths.

As observed from the data in FIG. 17D, the selection of the UV_{260/280} cutoff to end the wash phase significantly impacted the yield and purity of the isocratic elution pool. Using a cutoff of 0.7 to end the wash phase resulted in only 54.8% VC in the elution pool. In effect, this condition skipped the wash phase entirely, as UV_{260/280=0.70} was already achieved at the end of the loading phase. Allowing the wash to continue until UV_{260/280=1.00} resulted in a pool with 74.3% VC, and higher cutoffs resulted in continued improvements up to 94.9% VC using a cutoff of 1.40. The % genomic yield followed an inverse trend, with 91% yield obtained for a cutoff of 1.00, decreasing to 60.3% yield for a cutoff of 1.4. To meet a target of >80% VC with >80% genomic yield, a target cutoff of 1.15 with a range of 1.10-1.20 was selected for the wash phase.

### Example 9—Rapid Cycling Approach with Isocratic Weak Partitioning AEX

Isocratic weak partitioning AEX is very well-suited to operating in rapid cycling mode. One key reason is that the loading amount is the critical factor controlling the weak displacement. Thus, it is beneficial to have several cycles of operation such that the load can be cut-off at the right time without significant material loss. Sizing a monolith to operate for 10 or more cycles per batch would ensure that only a small percentage of material would remain un-loaded at the end, as well as accommodate batch-to-batch variability in bioreactor titers. While this would correspond to a small yield loss, the high step yields of 80% or more obtained using the optimized weak partitioning AEX strategy would still result in overall higher step yield than the standard low-loading process. The most ideal scenario would arise in the case of continuous operation, where a constant inflow of affinity-purified material would allow for hundreds of AEX cycles to be conducted with <1% material loss at the end of a weeks-long campaign.

Other advantages of operating for >10 cycles are that the monolith can be sized >100-fold smaller than those typically used for anion exchange separations of EC/VC, due to the combined effect of a 10-fold higher load and 10-fold increase in the number of cycles. This also allows the monolith to be operated at high flow rates of 5-10 CV/min, as the smaller size results in less system back-pressure. For example, though commercially available 4 L monoliths are rated for flowrates of up to 10 CV/min (40 L/min), even operating at 1 CV/min (4 L/min) results in ˜4 bar of system pressure, which is the maximum rating for manufacturing-scale chromatography equipment with single-use flow paths such as the AKTA Ready. In contrast, flowing a 40 mL monolith at 5-10 CV/min (200-400 mL/min) resulted in only 2-3 bar of system pressure. The isocratic elution phase is also only 10 CV in duration, compared to ˜100 CVs for linear gradients. Thus, 10 cycles can be conducted in approximately the same process time as a single cycle of linear gradient operation, with total process automation with the use of UV_{260/280}-based triggers to end the loading and wash phases.

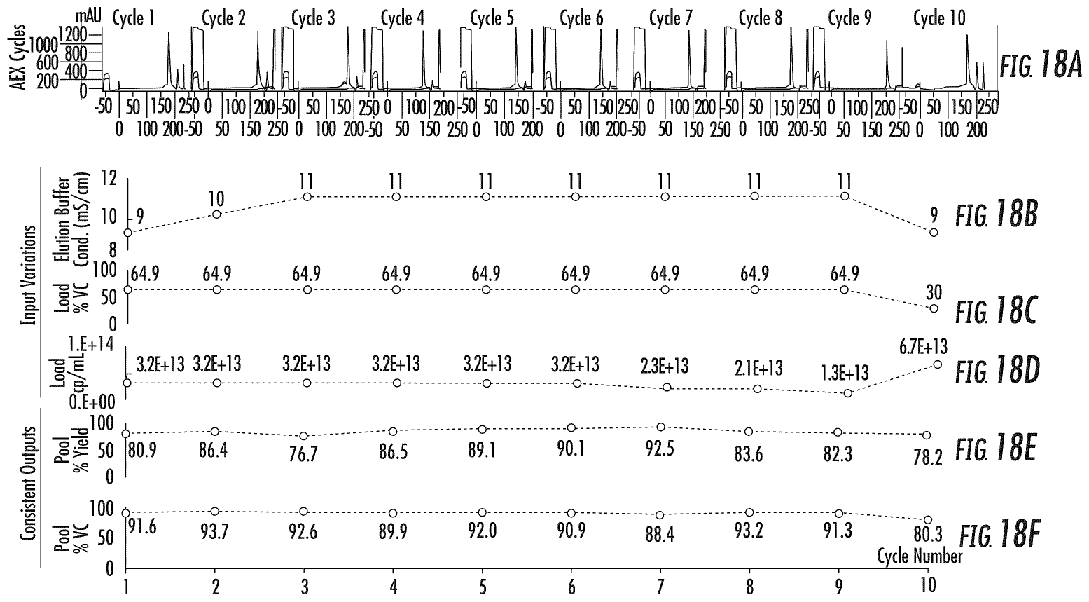

A 10-cycle test run with rapid cycling was executed and the results are summarized in FIGS. 18A to 18F. The Figures showing results from rapid-cycling weak partitioning AEX across 10 cycles of operation (FIG. 18A) with process variations in elution buffer conductivity (FIG. 18B), load % VC (FIG. 18C) and load capsid titer (FIG. 18D), resulting in consistently 75% or more genomic yield (FIG. 18E) and 80% or more VC (FIG. 18F) in the elution pools for the 3-hour rapid cycling process. FIG. 18A illustrates the 10 different elution chromatograms, all collected from the same 0.2 mL SpeCIMen CIM QA monolith taken from a 400 mL parent unit. The complete chromatogram is included for all runs, included pre-strip, loading, wash, isocratic elution, and post-strip phases. To further test robustness of the method, several process variations in buffer conductivity, load material % VC, and load material capsid titer were manually induced during the operation. These are detailed in FIGS. 18B, 18C and 18D: high-salt elution buffer conductivity varying from about 9 to 11 mS/cm (FIG. 18B), load % VC varying from about 30-65% (FIG. 18C), and load capsid titer varying from 1.3e13-6.7e13 cp/mL (FIG. 18D) by diluting or switching the load material and buffers. These variations were designed to cover a range of potential variations that could arise day-to-day during continuous operations, including lot-to-lot variability in buffers and changes in the load material characteristics.

Overall, the total 10-cycle batch resulted in combined pool % VC of about 90% with genomic yield of about 85%. The material was processed at a total monolith loading of 2e16 cp/mL This is equivalent to using a 40 mL scale monolith unit to process a 500 L scale upstream bioreactor with a high titer of 2e12 cp/mL. Process targets for this load material based on former studies using linear gradient AEX operations were 80% or more VC with 50% or more step yield. The rapid cycling weak partitioning AEX process exceeded purity targets by 10% and yield targets by 35%.

### Example 10—Comparison of Weak Partitioning and Linear Gradient AEX Shows Advantages of the Former Approach when Managing Growing Upstream Titers Across the Industry

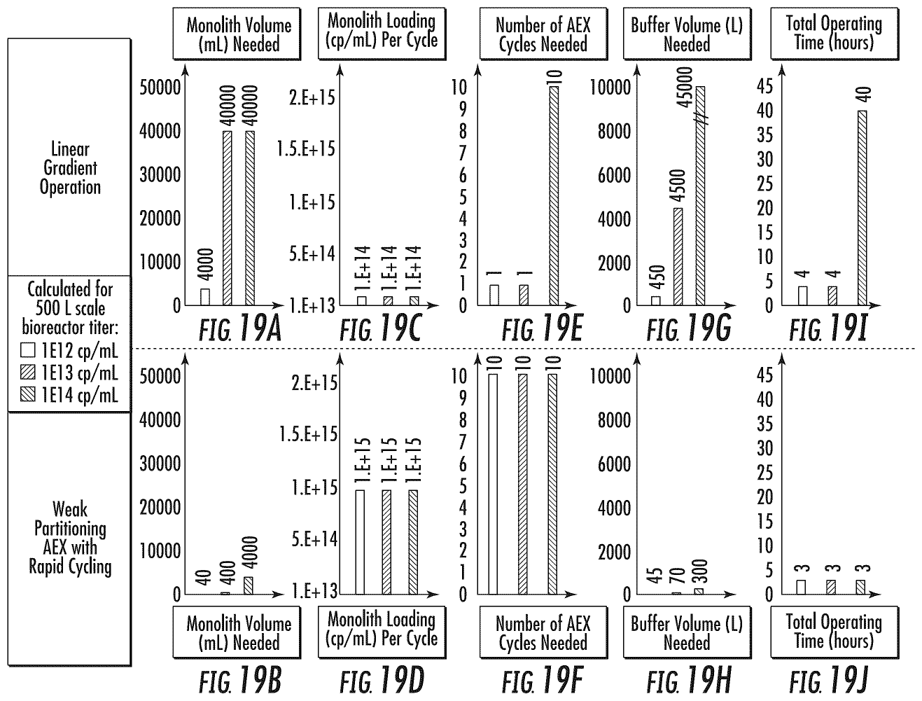

A point in favor of rapid-cycling weak partitioning AEX is that current industry-standard methods using linear gradients are approaching scalability limits, whereas weak partitioning approaches can comfortably be used to process 100-fold higher material. Linear gradients begin to present significant operational challenges with growing upstream bioreactor titers. This is illustrated in FIGS. 19A to 19J, which details the requirements for executing both linear gradient and rapid-cycling weak partitioning AEX for processing material from a 500 L upstream bioreactor with titers of 1e12, 1e13 and 1e14 cp/mL, respectively. Note that reported titers in the industry have already begin to exceed 1e12 cp/mL, which corresponds to 0.02 g/L of AAV capsid protein. With rapid advancements in AAV production techniques, it can be expected that titers can reach the 1e13-1e14 cp/mL level.

FIGS. 19A to 19J depict comparisons of operating parameters including monolith volume needed (FIGS. 19A-19B), monolith loading per cycle (FIGS. 19C-19D), number of cycles (FIGS. 19E-19F), buffer volume needed (FIGS. 19G-19H), and total operating time (FIGS. 19I-19J) to process 500 L scale bioreactors with titers in the range of 1e12 to 1e14 cp/mL using linear gradient operation (FIGS. 19A, 19C, 19E, 19G and 19I) compared to rapid cycling weak partitioning AEX (FIGS. 19B, 19D, 19F, 19H and 19J).

FIGS. 19A, 19C, 19E, 19G and 19I detail the process requirements for linear gradient AEX. With this approach, monolith loadings are required to be in the order of 1e14 cp/mL. A 500L scale bioreactor with 1e12 cp/mL titer is straightforward to process in 1 cycle on a 4 L monolith, requiring 450 L of buffer and 4 hours of operating time at a flow rate of 4 L/min. For managing a ten-fold titer increase to 1e13 cp/mL, a transition from a 4 L to a 40 L monolith is needed, with an associated increase in buffer requirement from 450 L to 4500 L presenting facility fit constraints. Note further that at present there is no available chromatography system with disposable flow-path that can operate at the high flow rates of 40 L/min that are required for these large-scale monoliths, posing challenges for the gene therapy field which is heavily based on single-use technology.

To maintain single-use standards, increasing cycles on a 4 L monolith may be preferred over switching to a 40 L monolith, which would increase processing time by 4 hours per cycle and buffer requirements by 450 L per cycle (up to 40 hours and 4500 L of buffer for 10 cycles). Finally, a further titer increase to 1e14 cp/mL would necessitate 10 cycles on a 40 L monolith, as manufacturing larger monoliths is not possible using standard approaches. This increases buffer requirements to 45000 L and operating time to 40 hours. Transitioning to resin-based AEX modalities can allow for larger columns to be packed, but these still require large buffer volumes equivalent to 100-fold of the column volume. Most AEX resins also have pore diffusion limitations that limit AAV separations, and flow at only 0.3-0.5 CV/min leading to lengthy processing times.

In contrast, FIGS. 19B, 19D, 19F, 19H and 19J detail the process requirements for rapid-cycling weak partitioning AEX. With this approach, monolith loadings are required to be in the order of 1e15 cp/mL. A 500 L scale bioreactor with 1e12 cp/mL titer can be processed using 10 cycles on a 40 mL monolith. This is a 100-fold reduction in monolith volume, combined with a 10-fold reduction in buffer requirements while maintaining total processing time of only 3 hours at a flow rate of 200 mL/min. A similar 10-cycle process can be implemented for upstream bioreactors with titers of 1e13 or 1e14 cp/mL by simply increasing the monolith volume to 400 mL or 4 L, while maintaining low buffer requirements of less than 300 L and processing time of 3 hours. Variations in bioreactor titer can be accommodated by varying the total number of cycles in the range of 5-15 or switching to the 80 mL/800 mL/8000 mL monolith series.

### Example 11—Rapid Cycling Weak Partitioning AEX Chromatography to Reduce Anion Exchange Monolith Volume Requirements

Partitioning AEX chromatography was used to purify 50 L of AAV8 harvest material in a rapid-cycling chromatography process suitable for continuous processing. The goal was to reduce anion exchange monolith volume requirements by >95% (from 400 mL monolith for a 50 L batch to 5 mL) by utilizing rapid cycling weak partitioning AEX, and simplify elution collection operations via isocratic elution by utilizing a single high-salt buffer to (a) condition the load material to 6-8 mS/cm and (b) create an isocratic elution step.

Method and components utilized include:
Column: 5×1 mL CIM QA HR monoliths;
System: Akta Avant 25 (Mario);
Loading: Controlled by UV_{260/280} cut-offs developed for weak partitioning AEX (loading until UV_{260/280}=0.7, followed by wash until UV_{260/280}:
Buffer A: 20 mM BTP, 3 mM MgCI2, 0.001% P188, pH 8.5;
Buffer B: 20 mM BTP, 100 mM NaCl, 3 mM MgCl2, 0.001% P188, pH 8.5;
Residence Time: 12 sec (5 monolith volumes per minute);
Total Cycles: 20 per monolith before switching to a new monolith; and
Total Operating Time: ˜20-30 minutes per cycle, ˜100 cycles total.

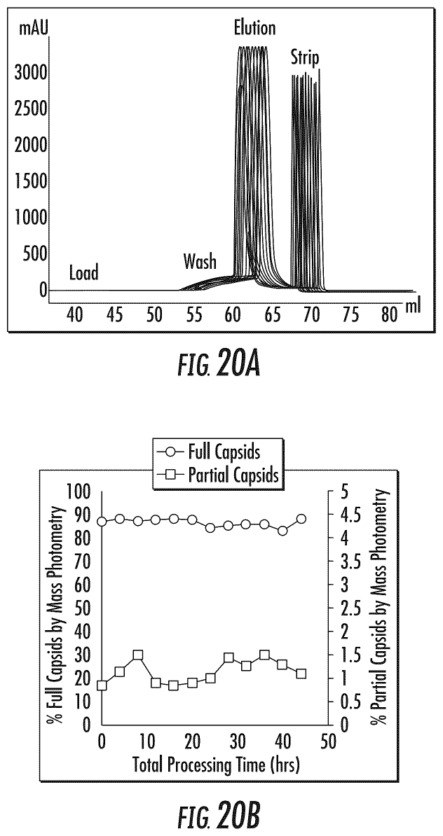

Chromatograms and analytical results are depicted FIGS. 20A to 20B. FIG. 20A shows the overlay of loading, wash, elution, and strip phases of 20 cycles of partitioning AEX on 1 mL CIM QA HR monolith. Percent (%) Full and % Partial capsids in partitioning AEX pool tracked over 42 hours of rapid cycling operation are shown in FIG. 20B.

Affinity captured pool from a 50 L batch of AAV8 harvest material was processed using 5×1 mL CIM QA monoliths over 42 hours of operation using rapid cycling weak partitioning AEX. The product pool measured 82.9-88.2% full and 0.85-1.5% partial capsids, exceeding purification targets of >75% full capsids and <10% partial capsids. The use of only 5×1 mL CIM QA HR monoliths represented a reduction of 98.75% in monolith volume requirements from the typical 400 mL CIM QA HR monolith that is required to process an equivalent amount of upstream material.

Overall, it has been demonstrated that weak partitioning AEX is a compelling alternative and improvement to standard linear gradient operations, with the potential to emerge as a platform for purification of next-generation, high-titer processes. Such processes are increasingly necessary to manage growing patient demand for innovative genetic therapies, particularly those requiring systemic administration and thus higher dosage.

It is to be understood that the description, specific examples, and data, while indicating exemplary embodiments, are given by way of illustration, and are not intended to limit the present inventions. Various changes and modifications within the present inventions, including combining embodiments in whole and in part, will become apparent to the skilled artisan from the discussion, disclosure and data contained herein, and thus are considered part of the inventions.

1. A method of purifying a preparation of recombinant adeno-associated virus (rAAV) particles that comprise a gene of interest (GOI), wherein the method comprises the steps of:
(a) loading a preparation comprising rAAV viral particles in a load buffer adjusted to a final load conductivity of about 2 to 12 mS/cm and pH of about 6.0 to about 11 on anion exchange chromatography (AEX) column, and continue the loading beyond the AEX column binding capacity until some AAV capsids flow out of the column while load material is still being flowed in, wherein the load amount is between about 1×10¹⁵ to 6×10¹⁵ cp/ml;
(b) eluting the column with an elution buffer, wherein the elution buffer comprises the load buffer and at least one salt to increase the conductivity relative to the load buffer; and
(c) collecting separated fractions of full and not full rAAV capsids using a linear gradient elution or an isocratic elution.

2. The method according to claim 1, wherein the conductivity and pH of the load buffer are adjusted prior to loading on the column.

3. The method according to claim 1, wherein the pH of the load buffer is about 6.0, 6.5, 7.0, 7.5, 8.0, 8.5, 9.0, 9.5, 10.0, 10.5, or 11.0.

4. The method according to claim 1, wherein the final load conductivity is about 2 mS/cm, about 3 mS/cm, about 4 mS/cm, about 5 mS/cm, about 6 mS/cm, about 7 mS/cm, about 8 mS/cm, about 9 mS/cm, about 10 mS/cm, or about 11 mS/cm.

5. The method according to claim 1, wherein genomic yield is about 20%, 25%, 30%, 35%, 40%, 45%, 50%, 55%, 60%, 65%, 70%, 75%, 80%, 85%, 90% or more with VC of 65% VC, 70% VC, 75% VC, 80% VC, 85% VC, 90% VC or more in the separated fractions.

6. The method according to claim 1, wherein the AEX is conducted in multiple cycles with rapid flow rates of about 1 to 10 CV/min, about 1 to 9 CV/min, about 1 to 8 CV/min, about 1 to 7 CV/min, about 1 to 6 CV/min, about 1 to 5 CV/min, about 1 to 4 CV/min, about 1 to 3 CV/min, or about 1 to 2 CV/min.

7. The method according to claim 1, wherein the rAAV viral particles are loaded on at least one AEX column, and wherein the AEX column unit is selected from the group consisting of monoliths, resins, gels, and membranes.

8. (canceled)

9. The method according to claim 1, wherein the salt is NaCl, KCl, MgCl₂, CaCl₂), NH₄Cl, Na₂SO₄, CaSO₄, K₂SO₄, MgSO₄, (NH₄)₂SO₄, sodium citrate, tetramethylammonium chloride (TMAC) or a mixture thereof.

10. The method according to claim 1, wherein non-full capsids elute before full capsids.

11. The method according to claim 1, wherein full capsids elute before non-full capsids.

12. A method of separating full capsids and empty capsids in a preparation of recombinant adeno-associated virus (rAAV) particles that comprise a gene of interest (GOI), wherein the method comprises the steps of:
(a) loading a preparation comprising rAAV viral particles in a load buffer adjusted to a final load conductivity of about 2 to 12 mS/cm and pH of about 6.0 to about 11 on anion exchange chromatography (AEX) column, and continue the loading beyond the AEX column binding capacity until some AAV capsids flow out of the column while load material is still being flowed in, wherein the load amount is between about 1×10¹⁵ to 6×10¹⁵ cp/ml;
(b) eluting the column with an elution buffer, wherein the elution buffer comprises the load buffer and at least one salt to increase the conductivity relative to the load buffer; and
(c) collecting separated fractions of full and not full rAAV capsids using a linear gradient elution or an isocratic elution.

13. The method according to claim 12, wherein the conductivity and pH of the preparation to be loaded are adjusted prior to loading on the column.

14. The method according to claim 12, wherein the pH of the load buffer is about 6.0, 6.5, 7.0, 7.5, 8.0, 8.5, 9.0, 9.5, 10.0, 10.5, or 11.0.

15. The method according to claim 12, wherein the final load conductivity is about 2 mS/cm, about 3 mS/cm, about 4 mS/cm, about 5 mS/cm, about 6 mS/cm, about 7 mS/cm, about 8 mS/cm, about 9 mS/cm, about 10 mS/cm, or about 11 mS/cm.

16. The method according to claim 12, wherein genomic yield is about 20%, 25%, 30%, 35%, 40%, 45%, 50%, 55%, 60%, 65%, 70%, 75%, 80%, 85%, 90% or more with VC of 65% VC, 70% VC, 75% VC, 80% VC, 85% VC, 90% VC or more in the separated fractions.

17. The method according to claim 12, wherein the AEX is conducted in multiple cycles with rapid flow rates of about 1 to 10 CV/min, about 1 to 9 CV/min, about 1 to 8 CV/min, about 1 to 7 CV/min, about 1 to 6 CV/min, about 1 to 5 CV/min, about 1 to 4 CV/min, about 1 to 3 CV/min, or about 1 to 2 CV/min.

18. The method according to claim 12, wherein the rAAV viral particles are loaded on at least one AEX column, and wherein the AEX column unit is selected from the group consisting of monoliths, resins, gels, and membranes.

19. (canceled)

20. The method according to claim 12, wherein the salt is NaCl, KCl, MgCl₂, CaCl₂), NH₄Cl, Na₂SO₄, CaSO₄, K₂SO₄, MgSO₄, (NH₄)₂SO₄, sodium citrate, tetramethylammonium chloride (TMAC) or a mixture thereof.

21. The method according to claim 12, wherein non-full capsids elute before full capsids.

22. The method according to claim 12, wherein full capsids elute before non-full capsids.

23. A purified preparation of recombinant adeno-associated virus (rAAV) comprising a gene of interest (GOI), wherein the purified preparation is made by the method comprising the steps of:
(a) loading a preparation comprising rAAV viral particles in a load buffer adjusted to a final load conductivity of about 2 to 12 mS/cm and pH of about 6.0 to about 11 on anion exchange chromatography (AEX) column, and continue the loading beyond the AEX column binding capacity until some AAV capsids flow out of the column while load material is still being flowed in, wherein the load amount is between about 1×10¹⁵ to 6×10¹⁵ cp/ml;
(b) eluting the column with an elution buffer, wherein the elution buffer comprises the load buffer and at least one salt to increase the conductivity relative to the load buffer; and
(c) collecting separated fractions of full and not full rAAV capsids using a linear gradient elution or an isocratic elution.

24. The purified preparation of rAAV according to claim 23, wherein the conductivity and pH of the preparation to be loaded are adjusted prior to loading on the column.

25. The purified preparation of rAAV according to claim 23, wherein the AEX is conducted in multiple cycles with rapid flow rates of about 1 to 10 CV/min, about 1 to 9 CV/min, about 1 to 8 CV/min, about 1 to 7 CV/min, about 1 to 6 CV/min, about 1 to 5 CV/min, about 1 to 4 CV/min, about 1 to 3 CV/min, or about 1 to 2 CV/min.

26. The purified preparation of rAAV according to claim 23, wherein the pH of the load buffer is about 6.0, 6.5, 7.0, 7.5, 8.0, 8.5, 9.0, 9.5, 10.0, 10.5, or 11.0.

27. The purified preparation of rAAV according to claim 23, wherein the final load conductivity is about 2 mS/cm, about 3 mS/cm, about 4 mS/cm, about 5 mS/cm, about 6 mS/cm, about 7 mS/cm, about 8 mS/cm, about 9 mS/cm, about 10 mS/cm, or about 11 mS/cm.

28. The purified preparation of rAAV according to claim 23, wherein genomic yield is about 20%, 25%, 30%, 35%, 40%, 45%, 50%, 55%, 60%, 65%, 70%, 75%, 80%, 85%, 90% or more with VC of 65% VC, 70% VC, 75% VC, 80% VC, 85% VC, 90% VC or more in the separated fractions.

29. The purified preparation of rAAV according to claim 23, wherein the rAAV viral particles are loaded on at least one AEX column, and wherein the AEX column unit is selected from the group consisting of monoliths, resins, gels, and membranes.

30. (canceled)

31. The purified preparation of rAAV according to claim 23, wherein the salt is NaCl, KCl, MgCl₂, CaCl₂), NH₄Cl, Na₂SO₄, CaSO₄, K₂SO₄, MgSO₄, (NH₄)₂SO₄, sodium citrate, tetramethylammonium chloride (TMAC) or a mixture thereof.

32. The purified preparation of rAAV according to claim 23, wherein non-full capsids elute before full capsids.

33. The purified preparation of rAAV according to claim 23, wherein full capsids elute before non-full capsids.
